(19) United States
(12) Patent Application Publication
(10) Pub. No.: US 2009/0062360 A1
(43) Pub. Date: Mar. 5, 2009

(54) Compounds Which Inhibit the Glycine Transporter and Uses Thereof

(76) Inventors: Steven Coulton, Essex (GB); Howard Robert Marshall, Essex (GB); David John Nash, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/282,677

(22) PCT Filed: Mar. 14, 2007
(86) PCT No.: PCT/EP2007/052414

§ 371 Date: Sep. 12, 2008

(30) Foreign Application Priority Data
Mar. 16, 2006 (GB) 0605412.6
Mar. 22, 2006 (GB) 0605827.5
Nov. 17, 2006 (GB) 0622995.9
Feb. 1, 2007 (GB) 0701947.4

(51) Int. Cl. A61K 31/4184; C07D 235/02

(52) U.S. Cl. 514/387; 548/301.4

(57) ABSTRACT

Compounds of formula (I) and salts and solvates thereof are provided:

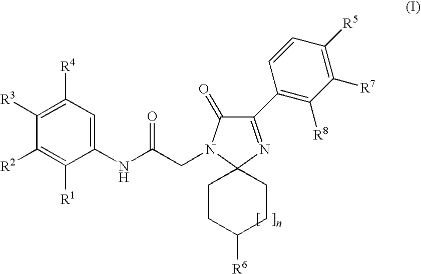

wherein R¹ to R⁸ and n are defined in the description. Uses of the compounds as medicaments, and in the manufacture of medicament for treating neurological and neuropsychiatric disorders, in particular psychoses, dementia or attention deficit disorder are also disclosed. The invention further comprises processes to make these compounds and pharmaceutical formulations thereof.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-Ia, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

A novel class of compounds which inhibit GlyT1 transporters have been found. The compounds are of potential use in the treatment of certain neurological and neuropsychiatric disorders, including schizophrenia.

Thus, in the first aspect, there is provided a compound of formula (I) or a salt or solvate thereof:

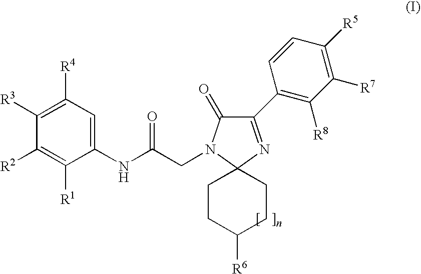

wherein:
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R³ is selected from H, methyl, ethyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
   or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R⁵ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
R⁷ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
   with the proviso that when R⁵ is selected from H, methyl, methoxy, chloro and fluoro, then R⁷ is not H;
R⁶ is selected from H and methyl;
R⁸ is selected from H and fluoro; and
n is selected from 0, 1 and 2.
“H” refers to hydrogen.

As used herein, the term “C₁-C₄alkyl” refers to a straight or branched alkyl group in all isomeric forms. Examples include methyl, ethyl, propyl, isopropyl, butyl, isobutyl, sec-butyl and tert-butyl.

As used herein, the term “C₁-C₄alkoxy” refers to the group —O—C₁-C₄alkyl wherein C₁-C₄alkyl is as defined above.

As used herein, the term “C₁-C₄alkoxyC₁-C₄alkyl” refers to the group (C₁₋₄alkyl)-O—(C₁₋₄alkyl), wherein C₁-C₄alkyl is as defined above.

As used herein, the term “C₃-C₆cycloalkyl” refers to a cycloalkyl group consisting of from 3 to 6 carbon atoms, ie cyclopropane, cyclobutane, cyclopentane or cyclohexane.

As used herein, the terms “halogen” and its abbreviation “halo” refer to fluorine, chlorine, bromine, or iodine.

As used herein, the term “haloC₁-C₄alkyl” refers to a C₁-C₄alkyl group as defined above which is substituted with any number of fluorine, chlorine, bromine, or iodine atoms, including with mixtures of those atoms. A haloC₁-C₄alkyl group may, for example contain 1, 2 or 3 halogen atoms. For example, a haloC₁-C₄alkyl group may have all hydrogen atoms replaced with halogen atoms. Examples of haloC₁-C₄alkyl groups include fluoromethyl, difluoromethyl and trifluoromethyl.

As used herein, the term “haloC₁-C₄alkoxy” refers to a C₁-C₄alkoxy group as defined above which is substituted with any number of fluorine, chlorine, bromine, or iodine atoms, including with mixtures of those atoms. A haloC₁-C₄alkoxy group may, for example contain 1, 2 or 3 halogen atoms. For example, a haloC₁-C₄alkoxy group may have all hydrogen atoms replaced with halogen atoms. Examples of haloC₁-C₄alkoxy groups include fluoromethyloxy, difluoromethyloxy and trifluoromethyloxy.

As used herein, the term “C₁-C₄alkylsulfonyl” refers to a group —SO₂(C₁-C₄alkyl). An example is —SO₂CH₃.

In one embodiment, R¹ is selected from H, C₁-C₂alkyl, C₁-C₂alkoxy, halo, haloC₁-C₂alkyl, haloC₁-C₂alkoxy, and cyano. In a further embodiment, R¹ is selected from H, methyl, methoxy and halo. In one embodiment, the halo group is selected from bromo, chloro and fluoro. In one embodiment, the halo group is selected from chloro and fluoro.

In one embodiment, R² is selected from H, C₁-C₂alkyl, C₁-C₂alkoxy, halo, haloC₁-C₂alkyl, haloC₁-C₂alkoxy, and cyano. In a further embodiment, R² is selected from H, methyl, halo, trifluoromethyl and cyano. In a further embodiment, R² is selected from H, methyl, trifluoromethyl, fluoro, cyano and bromo. In one embodiment, the halo group is selected from bromo, chloro and fluoro. In one embodiment, the halo group is selected from chloro and fluoro. In one embodiment the halo group is fluoro. In one embodiment, R² is fluoro. In one embodiment, R² is CF₃.

In one embodiment, R³ is selected from H, C₁-C₂alkyl, C₁-C₂alkoxy, halo, haloC₁-C₂alkyl, haloC₁-C₂alkoxy, and cyano. In a further embodiment, R³ is selected from H, methyl, methoxy, halo, and cyano. In one embodiment, the halo group is selected from bromo, chloro and fluoro. In one embodiment, the halo group is selected from chloro and fluoro. In one embodiment the halo group is fluoro.

In one embodiment, R⁴ is selected from H, C₁-C₂alkyl, C₁-C₂alkoxy, halo, haloC₁-C₂alkyl, haloC₁-C₂alkoxy, and cyano. In a further embodiment, R⁴ is selected from H, methyl, halo, and cyano. In a further embodiment, R⁴ is H. In one embodiment, R⁴ is fluoro.

In one embodiment, R⁵ is selected from H, C₁-C₄alkoxy, haloC₁-C₄alkyl, haloC₁-C₄alkoxy, halo, cyano, and C₁-C₄alkoxyC₁-C₄alkoxy. In one embodiment, R⁵ is selected from H, C₁-C₄alkoxy, haloC₁-C₄alkyl, haloC₁-C₄alkoxy, bromo, cyano, and C₁-C₄alkoxyC₁-C₄alkoxy.

In one embodiment, R⁵ is selected from H, C₁-C₂alkyl, C₁-C₂alkoxy, haloC₁-C₂alkyl, haloC₁-C₂alkoxy, halo, cyano, and C₁-C₂alkoxyC₁-C₂alkoxy. In one embodiment, R⁵ is selected from H, haloC₁-C₂alkyl, haloC₁-C₂alkoxy, C₁-C₂alkoxy, halo, cyano, and C₁-C₂alkoxyC₁-C₂alkoxy. In one embodiment, R⁵ is selected from H, haloC₁-C₂alkyl, haloC₁-C₂alkoxy, fluoro, bromo, chloro, cyano, and C₁-C₂alkoxyC₁-C₂alkoxy. In a further embodiment, R⁵ is selected from H, trifluoromethyl, trifluoromethoxy, bromo, cyano, and methoxyethoxy. In one embodiment, R⁵ is cyano.

In one embodiment, R⁷ is selected from H, C₁-C₂alkyl, C₁-C₂alkoxy, haloC₁-C₂alkyl, haloC₁-C₂alkoxy, halo, cyano, and C₁-C₂alkoxyC₁-C₂alkoxy; with the proviso that when R⁵ is selected from H, methyl, methoxy, chloro and fluoro, then R⁷ is not H.

In one embodiment, R⁵ is selected from H, methyl, methoxy, chloro and fluoro, and R⁷ is selected from C₁-C₂alkyl, C₁-C₂alkoxy, haloC₁-C₂alkyl, haloC₁-C₂alkoxy, halo, cyano, and C₁-C₂alkoxyC₁-C₂alkoxy.

In one embodiment, R⁷ is selected from C₁-C₂alkyl, C₁-C₂alkoxy, haloC₁-C₂alkyl, haloC₁-C₂alkoxy, halo, cyano, and C₁-C₂alkoxyC₁-C₂alkoxy.

In one embodiment, R⁷ is selected from H, cyano and halo, with the proviso that when R⁵ is selected from H, methyl, methoxy, chloro and fluoro, then R⁷ is not H. In a further embodiment, R⁷ is selected from H, chloro, bromo and cyano, with the proviso that when R⁵ is selected from H, methyl, methoxy, chloro and fluoro, then R⁷ is not H.

In one embodiment, R⁵ is selected from H, haloC₁-C₄alkyl, haloC₁-C₄alkoxy, bromo, cyano, and C₁-C₄alkoxyC₁-C₄alkoxy; and R⁷ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy; and R⁵ and R⁷ are not both H.

In one embodiment, R⁶ is H.

In one embodiment, n is selected from 0 and 1. In a further embodiment, n is 1.

In one embodiment, there is provided a compound of formula (Ia) or a salt or solvate thereof:

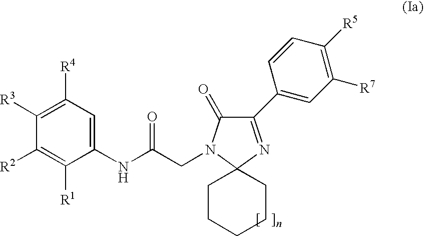

wherein:
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R³ is selected from H, methyl, ethyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
   or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R⁵ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
R⁷ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
   with the proviso that when R⁵ is selected from H, methyl, methoxy, chloro and fluoro, then R⁷ is not H;
n is selected from 0, 1 and 2.

The present invention also provides a compound of formula (Ib) or a salt or solvate thereof:

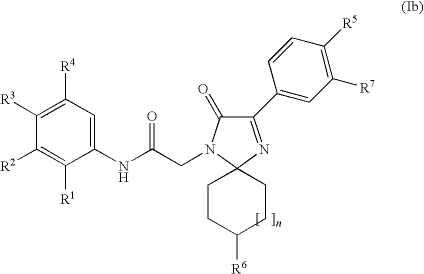

wherein:
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, and cyano;
R³ is selected from H, methyl, ethyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, and cyano;
   or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxy C₁-C₄alkyl, and cyano;
R⁵ is selected from H, haloC₁-C₄alkyl, haloC₁-C₄alkoxy, bromo, cyano, and C₁-C₄alkoxy C₁-C₄alkoxy;
R⁷ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
   and R⁵ and R⁷ are not both H;
R⁶ is selected from H and methyl; and
n is selected from 0, 1 and 2.

The present invention also provides a compound of formula (Ic) or a salt or solvate thereof:

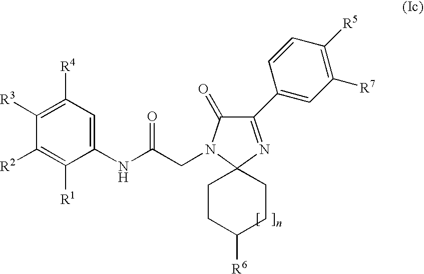

wherein:
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R³ is selected from H, methyl, ethyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
   or R² and R³ together form a group selected from —O—CH₂—O— and —O—CH₂—CH₂—O—;
R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl and cyano;
R⁵ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
R⁷ is selected from H, C₁-C₄alkyl, C₁-C₄alkoxy, haloC₁-C₄alkyl, haloC₁-C₄alkoxy, halo, cyano, and C₁-C₄alkoxyC₁-C₄alkoxy;
   with the proviso that when R⁵ is selected from H, methyl, methoxy, chloro and fluoro, then R⁷ is not H;
R⁶ is selected from H and methyl; and
n is selected from 0, 1 and 2.

The present invention also provides a compound of formula (Id) or a salt or solvate thereof:

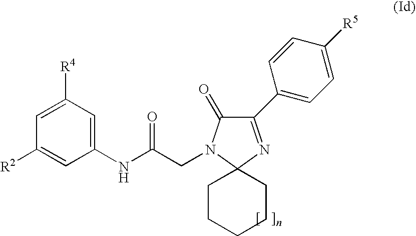

wherein:
R² is halo;
R⁴ is halo;
R⁵ is cyano; and
n is selected from 0 and 1.

In one embodiment, in formula (Id) above, R² and R⁴ are both fluoro.

The present invention also provides a compound of formula (Ie) or a salt or solvate thereof:

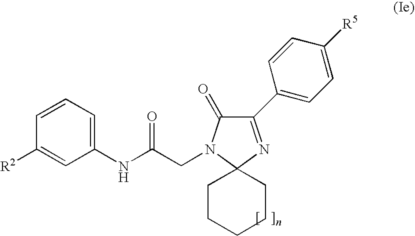

wherein:
R² is trifluoromethyl;
R⁵ is cyano; and
n is selected from 0 and 1.

All features and embodiments of formula (I) apply to formulae (Ia) to (Ie) mutatis mutandis. For the avoidance of doubt, the embodiments of any one feature of the compounds of the invention may be combined with any embodiment of another feature of compounds of the invention to create a further embodiment.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1S)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include water, ethanol and acetic acid. In one embodiment, the solvent used is water.

Examples of compounds of the invention include:
N-[2-(Methyloxy)phenyl]-2-{2-oxo-3-[4-(trifluoromethyl)phenyl]-1,4-diazaspiro[4.5]dec-3-en-1-yl}acetamide;
2-[3-(4-Bromophenyl)-2-oxo-1,4-diazaspiro[4.5]dec-3-en-1-yl]-N-(3,4-difluorophenyl)acetamide;
2-[3-(4-Cyanophenyl)-2-oxo-1,4-diazaspiro[4.5]dec-3-en-1-yl]-N-(3,4-difluorophenyl)acetamide;
N-(3-methylphenyl)-2-{2-oxo-3-[4-(trifluoromethyl)phenyl]-1,4-diazaspiro[4.5]dec-3-en-1-yl}acetamide;
N-(3,4-difluorophenyl)-2-{2-oxo-3-[4-(trifluoromethyl)phenyl]-1,4-diazaspiro[4.5]dec-3-en-1-yl}acetamide;
N-(3,4-difluorophenyl)-2-(2-oxo-3-{4-[(trifluoromethyl)oxy]phenyl}-1,4-diazaspiro[4.5]dec-3-en-1-yl)acetamide;
N-(2,4-dimethylphenyl)-2-(2-oxo-3-{4-[(trifluoromethyl)oxy]phenyl}-1,4-diazaspiro[4.5]dec-3-en-1-yl)acetamide;
N-[2-(methyloxy)phenyl]-2-(2-oxo-3-{4-[(trifluoromethyl)oxy]phenyl}-1,4-diazaspiro[4.5]dec-3-en-1-yl)acetamide;
2-[3-(3-chlorophenyl)-2-oxo-1,4-diazaspiro[4.5]dec-3-en-1-yl]-N-(3,4-difluorophenyl)acetamide;
2-[3-(3-chlorophenyl)-2-oxo-1,4-diazaspiro[4.5]dec-3-en-1-yl]-N-(2-methylphenyl)acetamide;
2-[3-(3-chlorophenyl)-2-oxo-1,4-diazaspiro[4.5]dec-3-en-1-yl]-N-(2,4-dimethylphenyl)acetamide;
2-[3-(3-chlorophenyl)-2-oxo-1,4-diazaspiro[4.5]dec-3-en-1-yl]-N-[2-(methyloxy)phenyl]acetamide;
2-[3-(4-bromophenyl)-2-oxo-1,4-diazaspiro[4.5]dec-3-en-1-yl]-N-(2,4-dimethylphenyl)acetamide;
N-(3,4-difluorophenyl)-2-[3-(4-{[2-(methyloxy)ethyl]oxy}phenyl)-2-oxo-1,4-diazaspiro[4.5]dec-3-en-1-yl]acetamide;
   and salts and solvates thereof.

Further examples include:
2-[3-(4-Cyanophenyl)-2-oxo-1,4-diazaspiro[4.4]non-3-en-1-yl]-N-(3,5-difluorophenyl)acetamide;
N-(2-Chlorophenyl)-2-{2-oxo-3-[4-(trifluoromethyl)phenyl]-1,4-diazaspiro[4.4]non-3-en-1-yl}acetamide;
N-(3,4-Difluorophenyl)-2-{2-oxo-3-[4-(trifluoromethyl)phenyl]-1,4-diazaspiro[4.4]non-3-en-1-yl}acetamide;
N-(3,5-Difluorophenyl)-2-{2-oxo-3-[4-(trifluoromethyl)phenyl]-1,4-diazaspiro[4.4]non-3-en-1-yl}acetamide;
N-[3,5-bis(trifluoromethyl)phenyl]-2-{2-oxo-3-[4-(trifluoromethyl)phenyl]-1,4-diazaspiro[4.5]dec-3-en-1-yl}acetamide;
N-[3-cyano-5-(trifluoromethyl)phenyl]-2-{2-oxo-3-[4-(trifluoromethyl)phenyl]-1,4-diazaspiro[4.5]dec-3-en-1-yl}acetamide;
N-[3-bromo-5-(trifluoromethyl)phenyl]-2-{2-oxo-3-[4-(trifluoromethyl)phenyl]-1,4-diazaspiro[4.5]dec-3-en-1-yl}acetamide;
N-(4-chloro-3-methylphenyl)-2-{2-oxo-3-[4-(trifluoromethyl)phenyl]-1,4-diazaspiro[4.5]dec-3-en-1-yl}acetamide;
N-(3-fluoro-5-methylphenyl)-2-{2-oxo-3-[4-(trifluoromethyl)phenyl]-1,4-diazaspiro[4.5]dec-3-en-1-yl}acetamide;
N-(2-fluoro-5-methylphenyl)-2-{2-oxo-3-[4-(trifluoromethyl)phenyl]-1,4-diazaspiro[4.5]dec-3-en-1-yl}acetamide;
N-(4-fluoro-3-methylphenyl)-2-{2-oxo-3-[4-(trifluoromethyl)phenyl]-1,4-diazaspiro[4.5]dec-3-en-1-yl}acetamide;
2-[3-(4-cyanophenyl)-2-oxo-1,4-diazaspiro[4.5]dec-3-en-1-yl]-N-(3,5-difluorophenyl)acetamide;
N-(3,5-difluorophenyl)-2-(2-oxo-3-{4-[(trifluoromethyl)oxy]phenyl}-1,4-diazaspiro[4.5]dec-3-en-1-yl)acetamide;
2-(2-oxo-3-{4-[(trifluoromethyl)oxy]phenyl}-1,4-diazaspiro[4.5]dec-3-en-1-yl)-N-(3,4,5-trifluorophenyl)acetamide;
2-(2-oxo-3-{4-[(trifluoromethyl)oxy]phenyl}-1,4-diazaspiro[4.5]dec-3-en-1-yl)-N-(2,3,5-trifluorophenyl)acetamide;
2-[3-(4-cyanophenyl)-2-oxo-1,4-diazaspiro[4.5]dec-3-en-1-yl]-N-(3,4,5-trifluorophenyl)acetamide;
N-(4-bromo-3,5-difluorophenyl)-2-[3-(4-cyanophenyl)-2-oxo-1,4-diazaspiro[4.5]dec-3-en-1-yl]acetamide;
N-(2-chloro-3,5-difluorophenyl)-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.5]dec-3-en-1-yl]-N-[3,5-difluoro-4-(trifluoromethyl)phenyl]acetamide;
2-[3-(4-cyanophenyl)-2-oxo-1,4-diazaspiro[4.5]dec-3-en-1-yl]-N-[3,5-difluoro-4-(methyloxy)phenyl]acetamide;
N-(4-cyano-3,5-difluorophenyl)-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.4]non-3-en-1-yl]-N-(3,4-difluorophenyl)acetamide;
2-[3-(4-cyanophenyl)-2-oxo-1,4-diazaspiro[4.5]dec-3-en-1-yl]-N-(2,4-dimethylphenyl)acetamide;
2-{3-[3-bromo-4-(methyloxy)phenyl]-2-oxo-1,4-diazaspiro[4.5]dec-3-en-1-yl}-N-(3,5-difluorophenyl)acetamide;
   and salts and solvates thereof.

Further examples include:
2-{3-[3-cyano-4-(methyloxy)phenyl]-2-oxo-1,4-diazaspiro[4.5]dec-3-en-1-yl}-N-(3,5-difluorophenyl)acetamide
2-[3-(4-cyanophenyl)-2-oxo-1,4-diazaspiro[4.4]non-3-en-1-yl]-N-[3-(trifluoromethyl)phenyl]acetamide
2-[3-(4-cyanophenyl)-2-oxo-1,4-diazaspiro[4.5]dec-3-en-1-yl]-N-[3-(trifluoromethyl)phenyl]acetamide
2-[3-(4-cyanophenyl)-2-oxo-1,4-diazaspiro[4.4]non-3-en-1-yl]-N-[4-fluoro-3-(trifluoromethyl)phenyl]acetamide
N-[2-chloro-3-(trifluoromethyl)phenyl]-2-[3-(4-cyanophenyl)-2-oxo-1,4-diazaspiro[4.4]non-3-en-1-yl]acetamide
N-[4-chloro-3-(trifluoromethyl)phenyl]-2-[3-(4-cyanophenyl)-2-oxo-1,4-diazaspiro[4.4]non-3-en-1-yl]acetamide
2-[3-(4-cyanophenyl)-2-oxo-1,4-diazaspiro[4.5]dec-3-en-1-yl]-N-[4-fluoro-3-(trifluoromethyl)phenyl]acetamide
N-[2-chloro-3-(trifluoromethyl)phenyl]-2-[3-(4-cyanophenyl)-2-oxo-1,4-diazaspiro[4.5]dec-3-en-1-yl]acetamide
2-[3-(4-cyanophenyl)-2-oxo-1,4-diazaspiro[4.4]non-3-en-1-yl]-N-[3-fluoro-5-(trifluoromethyl)phenyl]acetamide
2-[3-(4-cyanophenyl)-2-oxo-1,4-diazaspiro[4.5]dec-3-en-1-yl]-N-[3-fluoro-5-(trifluoromethyl)phenyl]acetamide
2-[3-(4-cyanophenyl)-2-oxo-1,4-diazaspiro[4.4]non-3-en-1-yl]-N-[2-methyl-5-(trifluoromethyl)phenyl]acetamide
2-[3-(4-cyanophenyl)-2-oxo-1,4-diazaspiro[4.5]dec-3-en-1-yl]-N-[2-methyl-5-(trifluoromethyl)phenyl]acetamide
N-[4-chloro-3-(trifluoromethyl)phenyl]-2-[3-(4-cyanophenyl)-2-oxo-1,4-diazaspiro[4.5]dec-3-en-1-yl]acetamide
N-[2-chloro-5-(trifluoromethyl)phenyl]-2-[3-(4-cyanophenyl)-2-oxo-1,4-diazaspiro[4.4]non-3-en-1-yl]acetamide
N-[2-chloro-5-(trifluoromethyl)phenyl]-2-[3-(4-cyanophenyl)-2-oxo-1,4-diazaspiro[4.5]dec-3-en-1-yl]acetamide
N-(3-cyanophenyl)-2-[3-(4-cyanophenyl)-2-oxo-1,4-diazaspiro[4.4]non-3-en-1-yl]acetamide
N-(3-cyanophenyl)-2-[3-(4-cyanophenyl)-2-oxo-1,4-diazaspiro[4.5]dec-3-en-1-yl]acetamide
N-(3-cyano-4-fluorophenyl)-2-[3-(4-cyanophenyl)-2-oxo-1,4-diazaspiro[4.5]dec-3-en-1-yl]acetamide
N-(3-cyano-5-fluorophenyl)-2-[3-(4-cyanophenyl)-2-oxo-1,4-diazaspiro[4.5]dec-3-en-1-yl]acetamide
N-(3-cyano-4-methylphenyl)-2-[3-(4-cyanophenyl)-2-oxo-1,4-diazaspiro[4.5]dec-3-en-1-yl]acetamide
2-[3-(4-cyanophenyl)-2-oxo-1,4-diazaspiro[4.4]non-3-en-1-yl]-N-{3-[(difluoromethyl)oxy]phenyl}acetamide
2-[3-(4-cyanophenyl)-2-oxo-1,4-diazaspiro[4.4]non-3-en-1-yl]-N-{3-[(trifluoromethyl)oxy]phenyl}acetamide
2-[3-(4-cyanophenyl)-2-oxo-1,4-diazaspiro[4.4]non-3-en-1-yl]-N-[3-(difluoromethyl)phenyl]acetamide
2-[3-(4-cyanophenyl)-2-oxo-1,4-diazaspiro[4.4]non-3-en-1-yl]-N-{3-[(methyloxy)methyl]phenyl}acetamide
2-[3-(4-cyanophenyl)-2-oxo-1,4-diazaspiro[4.4]non-3-en-1-yl]-N-(2,4-dimethylphenyl)acetamide
2-[3-(4-cyanophenyl)-2-oxo-1,4-diazaspiro[4.4]non-3-en-1-yl]-N-(3-methylphenyl)acetamide
2-[3-(4-cyanophenyl)-2-oxo-1,4-diazaspiro[4.5]dec-3-en-1-yl]-N-{3-[(difluoromethyl)oxy]phenyl}acetamide
2-[3-(4-cyanophenyl)-2-oxo-1,4-diazaspiro[4.5]dec-3-en-1-yl]-N-{3-[(trifluoromethyl)oxy]phenyl}acetamide
2-[3-(4-cyanophenyl)-2-oxo-1,4-diazaspiro[4.5]dec-3-en-1-yl]-N-[3-(difluoromethyl)phenyl]acetamide
2-[3-(4-cyanophenyl)-2-oxo-1,4-diazaspiro[4.5]dec-3-en-1-yl]-N-{3-[(methyloxy)methyl]phenyl}acetamide
   and salts and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protectinq Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

In the following processes, the substituents have the same meanings as for formula (I) unless otherwise stated.

In another aspect, the present invention provides a process for the manufacture of a compound of formula (I) as defined above, the process comprising:

(a) reacting a compound of formula (II):

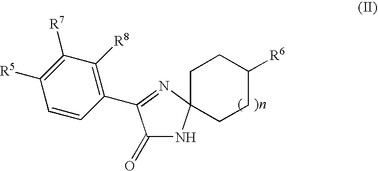

wherein R⁵, R⁷, R⁶, R⁸ and n are as defined for formula (I), with a compound of formula (III):

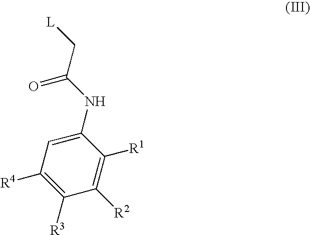

wherein R¹, R², R³, R⁴ and are as defined for formula (I), and L is a leaving group;
or
(b) reacting a compound of formula (XV):

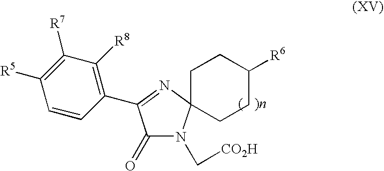

wherein R⁵, R⁷, R⁶, R⁸ and n are as defined for formula (I), with a compound of formula (IV):

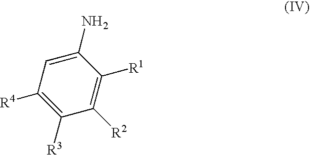

wherein R¹, R², R³ and R⁴ are as defined for formula (I);
or
(c) reacting a compound of formula (XVI):

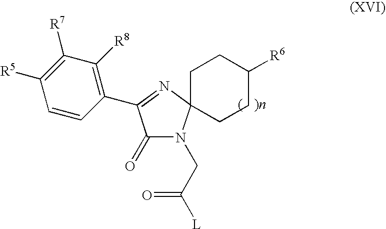

wherein R⁵, R⁶, R⁷ and n are as defined in formula (I) and L represents a leaving group; with a compound of formula (IV) as defined in process (b);
and thereafter optionally:
removing any protecting groups and/or
converting a compound of formula (I) into another compound of formula (I) and/or
forming a salt or solvate.

For process (a), a compound of formula (II) may be reacted with a base, for example sodium hydride, in a suitable inert solvent, for example dimethylformamide, followed by treatment with a compound of formula (III).

For process (b), compounds of formula (XV) can be converted to compounds of formula (I) by reaction with an aniline of formula (XVI) using a variety of methods known in the art. For example, step (vi) can be achieved by reaction of the acid (XV) with an aniline of formula (XVI), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU).

For process (c), examples of L include halogen, OC(═O)alkyl, OC(═O)O-alkyl and OSO₂Me. In one embodiment, L is halogen and the process is carried out in an inert solvent such as dichloromethane, in the presence of a base, such as triethylamine.

Compounds of formula (III) can be prepared by standard methods, for example as shown in Scheme 1. For example an aniline of formula (IV) may be combined with chloroacetyl chloride in an inert solvent, for example dioxan, and heated to give a compound of formula (III).

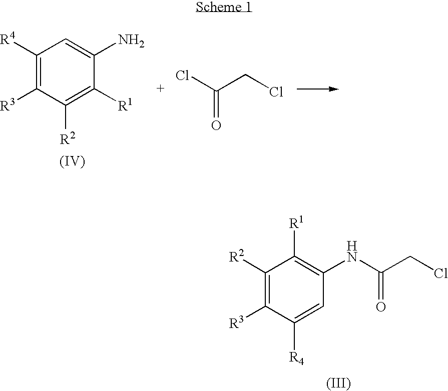

Compounds of formula (II) may be prepared by desulphurisation of compounds of formula (V) using an oxidising agent, for example hydrogen peroxide as shown for example in Scheme 2.

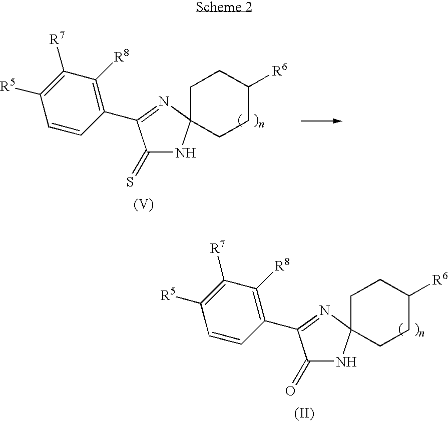

Compounds of formula (V) can be prepared by treating a ketothioamide of formula (VI) with the appropriate ketone of formula (VII) in the presence of a source of ammonia, for example ammonium acetate as shown in Scheme 3. In one embodiment, this reaction is performed in a solvent, for example isopropanol, at room or elevated temperature, preferably elevated temperature, for example at reflux.

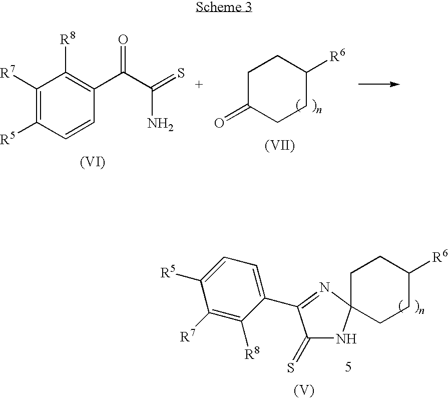

Thioamides of formula (VI) can be prepared from acylnitriles of formula (VIII) by treating with, for example hydrogen sulphide in the presence of an organic base, for example triethylamine in an inert solvent, for example diethyl ether at room temperature, as shown in Scheme 4. Acylnitriles of formula (VIII) can be prepared from the appropriate acid chloride (IX) and a source of cyanide, conveniently copper (I) cyanide, at elevated temperatures, for example greater than 150° C. preferably in the absence of solvent.

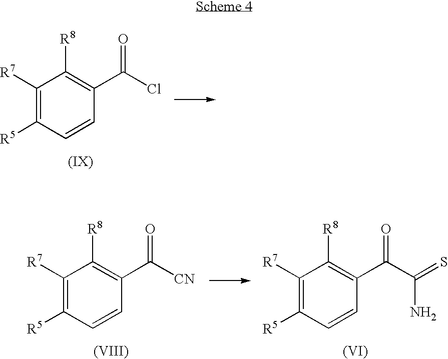

Alternatively, compounds of formula (II) can be synthesised as shown in Scheme 5.

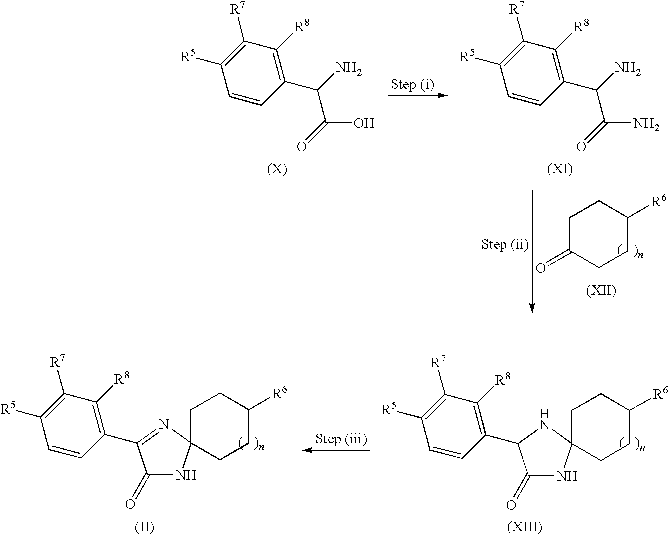

wherein R⁵, R⁶ and R⁷ are as defined for formula (I).

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XIII), step (ii), by condensation with ketones of formula (XII), for example, by heating in an inert solvent such as methanol, in the presence or absence of a catalyst such as H—Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (XV) may be prepared as shown in Scheme 6.

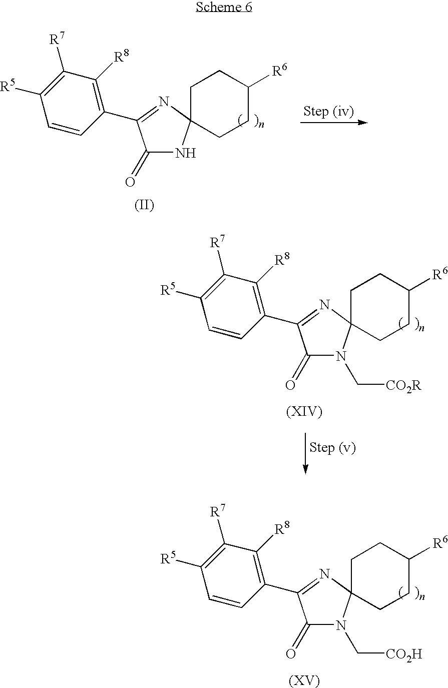

wherein R¹, R², R³, R⁴, R⁵, R⁶, R⁷ and R⁸ are as defined for compounds of formula (I).

Compounds of formula (XIV) can be prepared using standard methods from compounds of formula (II), step (iv), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (XIV) to afford the acids of formula (XV), step (v), can be achieved by known methods, for example by use of a base, such as sodium hydroxide, in an inert solvent, such as aqueous methanol or aqueous ethanol, with or without heating as appropriate.

Compounds of formula (XVI) may be prepared from compounds of formula (XV) using conventional chemistry.

Compounds of formula (I) can be converted into further compounds of formula (I) using standard techniques. For example, a group R¹ may be converted into another group R¹ and similarly groups R², R³, R⁴, R⁵, R⁶ and R⁷ using conventional chemistry. Salts may be prepared conventionally by reaction with the appropriate acid or acid derivative.

The compounds of the present invention inhibit the GlyT1 transporter. The compounds may selectively inhibit the GlyT1 transporter over the GlyT2 transporter. Some compounds of the invention may have mixed GlyT-1/GlyT-2 activity.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assays.

1) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. plC₅₀ values were determined using ActivityBase.

2) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37 C and 5% CO2. Cells grown to 70-80% confluency in T175 flasks were harvested and frozen. For the assay, cells were defrosted and resuspended at 1.32×106 cells/mL in assay buffer [140 mM NaCl, 5.4 mM KCl, 1.8 mM CaCl2, 0.8 mM MgSO4, 20 mM HEPES, 5 mM glucose and 5 mM alanine, pH 7.4]. Compounds were serially diluted 4-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension (1.32×106) and 5 uL of the cell/bead suspension transferred to each well of a LV384-well white solid bottom plate (3300 cells/well) containing 100 nL of test compounds. Substrate (5 uL) was added to each well [1:100 dilution of [3H]-glycine stock in assay buffer containing 2.5 uM glycine). Final DMSO concentration was 1% v/v. Data was collected using a Perkin Elmer Viewlux. plC50 values were determined using ActivityBase.

Compounds may have activity at the GlyT1 transporter if they have a plC₅₀ of 5.0 or above. Example compounds 1-59 below were found to have a plC₅₀ at the GlyT1 transporter of equal to greater than 6.0. Some compounds of the invention were found to have a plC₅₀ at the GlyT1 transporter of greater than 7.0.

Accordingly, in one aspect of the invention, there is provided a compound of formula (I) as hereinbefore described or a salt or solvate thereof, for use as a medicament. In a further aspect of the invention, there is provided a compound of formula (I) as hereinbefore described or a salt or solvate thereof, for use in the treatment of a disorder mediated by GlyT1.

In order to use a compound of the present invention as a medicament, it will normally be formulated into a pharmaceutical composition in accordance with standard pharmaceutical practice. The present invention also provides a pharmaceutical composition, which comprises a compound of formula (I) or a salt or solvate thereof, and a carrier, diluent or excipient.

In a further aspect, the present invention provides a process for preparing a pharmaceutical composition, the process comprising mixing a compound of formula (I) or a salt or solvate thereof and a carrier, diluent or excipient.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) may be also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) may also be of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of formula (I) may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of formula (I) may also be of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt or solvate thereof. Treatment of epilepsy may be carried out by the administration of a non-toxic anticonvulsant effective amount of a compound of the formula (I) or a salt or solvate thereof.

The compounds of formula (I) may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

Other disorders include benign forgetfulness, childhood learning disorders and closed head injury, Parkinson's disease, dyskinetic disorders, cognitive impairment, emesis, movement disorders, amnesia, circadian rhythm disorders, aggression and vertigo.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

In another aspect of the invention, there is provided use of a compound of formula (I) as hereinbefore defined or a salt or solvate thereof in the preparation of a medicament for the treatment of a disorder mediated by GlyT1.

In one embodiment, the disorder mediated by GlyT1 to be treated by the use or method as hereinbefore described is a psychosis (including schizophrenia), dementia or an attention deficit disorder. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

The compounds of formula (I) and their salts and solvates thereof may also be suitable for combination with other active ingredients, such as typical and atypical antipsychotics, to provide improved treatment of psychotic disorders.

Thus, the present invention also provides:
i) a combination product comprising a compound of the invention and an antipsychotic;
ii) a pharmaceutical composition comprising a combination product as defined in i) above and at least one carrier, diluent or excipient;
iii) the use of a combination product as defined in i) above in the manufacture of a medicament for treating a disease or condition caused by a reduction or imbalance in glutamate receptor function in a mammal;
iv) a combination product as defined in i) above for use in treating a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination product as defined in i) above for use as a medicament;
vii) a method of treatment of a disease or condition caused by a reduction or imbalance in glutamate receptor function in a mammal comprising administering an effective amount of a combination product as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt or solvate thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt or solvate thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt or solvate thereof to a patient receiving therapeutic administration of at least one antipsychotic agent. In a further aspect, the invention provides the use of compounds of formula (I) or a salt or solvate thereof in the manufacture of a medicament for adjunctive therapeutic administration for the treatment of a psychotic disorder in a patient receiving therapeutic administration of at least one antipsychotic agent. The invention further provides compounds of formula (I) or a salt or solvate thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt or solvate thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt or solvate thereof. The invention further provides at least one antipsychotic agent for adjunctive therapeutic administration for the treatment of a psychotic disorder in a patient receiving therapeutic administration of compounds of formula (I) or a salt or solvate thereof.

In a further aspect, the invention provides a method of treatment of a psychotic disorder by simultaneous therapeutic administration of compounds of formula (I) or a salt or solvate thereof in combination with at least one antipsychotic agent. The invention further provides the use of a combination of compounds of formula (I) or a salt or solvate thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt or solvate thereof and at least one mood stabilising or antimanic agent, a pharmaceutical composition comprising compounds of formula (I) or a salt or solvate thereof and at least one mood stabilising or antimanic agent, the use of a pharmaceutical composition comprising compounds of formula (I) or a salt or solvate thereof and at least one mood stabilising or antimanic agent in the manufacture of a medicament for the treatment of a psychotic disorder, and a pharmaceutical composition comprising compounds of formula (I) or a salt or solvate thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Formulations suitable for rectal administration may be provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, such as about 5 to about 500 mg, or about 10 to about 100 mg of the active ingredient per unit dose which could be administered, for example, 1 to 4 times per day.

The invention is further illustrated by the following non-limiting examples.

### Abbreviations:

s.g. specific gravity
THF tetrahydrofuran
DCM dichloromethane
DMF dimethylformamide
NMP N-methylpyrrolidinone
iPrOH isopropyl alcohol
HATU O-(7-Azabenzotriazol-1-yl)-N,N,N′, N′-tetramethyluronium hexafluorophosphate

### Analytical LC/MS Chromatography Conditions:

### Method A

Column: Waters Atlantis 50 mm×4.6 mm, 3 um particle size
Mobile phase: A: 0.05% Formic acid+Water
B: Acetonitrile+0.05% Formic acid
   Gradient: 5-min runtime: 3% B to 97% B over 4 min
   Flow rate: 3 ml/min
   UV wavelength range: 220-330 nm

### Temperature: 30° C.

### Method B

Column: Waters Acquity 50 mm×2.1 mm, 1.7 um particle size
Mobile phase: A: Water+0.05% Formic acid
B: Acetonitrile+0.05% Formic acid
   Gradient: 2-min runtime: 3% B to 97% B over 1.3 min
   Flow rate: 1 ml/min
   UV wavelength range: 220-330 nm

### Temperature: 30° C.

### Preparative HPLC Conditions:

Preparative HPLC refers to methods where the material was purified by High Performance Liquid Chromatography on a Supelcosil ABZ+Plus 5 um column (10 cm×21.2 mm); Eluting solvents are: water (containing 0.1% TFA) (A) and acetonitrile (containing 0.1% TFA) (B); 10 minute runtime with a gradient elution of 30-85% B at a flow rate of 8 mL/min and UV detection at 254 nm.

MDAP (mass-directed automated preparation) refers to purification by HPLC, wherein fraction collection is triggered by detection of the programmed mass ion for the compound of interest.

### Mass Directed Auto-Purification System Chromatography Conditions:

### Method 1

Column: Waters Atlantis 19 mm×100 mm or 30 mm×100 mm, 5 um particle size
Mobile phase: A: 0.1% Formic acid+Water
   B: Acetonitrile+0.1% Formic acid
Gradient: 13.5 min runtime with 10 min gradient dependant on analytical retention time
Flow rate: 20 or 40 ml/min

### Method 2

Detection is by UV and fraction collection is triggered by observation of the programmed mass ion for the compound of interest. Software used is Micromass Masslynx version 4.0. The column used is typically a Supelco LCABZ++column whose dimensions are 20 mm internal diameter by 100 mm in length. The stationary phase particle size is 5 um; Eluting solvents are: water+0.1% formic acid (solvent A) and acetonitrile:water 95:5+0.05% formic acid (Solvent B); There are five methods used depending on the analytical retention time of the compound of interest. Each has a 15-minute runtime, which comprises of a 10-minute gradient followed by a 5-minute column flush and re-equilibration step; MDP 1.5-2.2=0-30%; MDP 2.0-2.8=5-30%; MDP 2.5-3.0=15-55% B; MDP 2.8-4.0=30-80% B; MDP 3.8-5.5=50-90% B; Flow rate of 20 mL/min.

In general, where purifications involve the use of MDAP, Method 1 is employed unless otherwise stated.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to. All reactions were either carried out under argon or may be carried out under argon, unless otherwise stated.

### DESCRIPTIONS AND EXAMPLES

### Description 1

### Methyl amino[4-(trifluoromethyl)phenyl]acetate hydrochloride

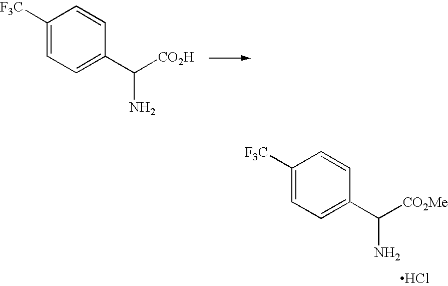

Thionyl chloride (5 ml; 68.9 mmol; 1.5 eq) was added dropwise under argon to methanol (100 ml) chilled in an ice-bath over 45 min. 4-Trifluoromethylphenylglycine (10 g; 45.6 mmol) was added and the mixture heated at 40° C. for 40 h. After cooling to room temperature, the reaction was evaporated under reduced pressure. The resulting solid was dissolved in methanol (200 ml) and evaporated under reduced pressure. Diethylether (250 ml) was added and the product filtered and dried to afford the title compound as the hydrochloride salt (12 g; 98%). ¹H NMR (d₆-DMSO) δ: 3.74 (3H, s), 5.52 (1H, s), 7.74 (2H, d, J=8 Hz), 7.89 (2H, d, J=8 Hz), 9.00 (3H, br s). Mass Spectrum (Electrospray LC/MS) Found 234 (MH⁺). C₁₀H₁₀F₃NO₂ requires 233. Ret. time 1.55 min.

### Description 2

### 2-Amino-2-[4-(trifluoromethyl)phenyl]acetamide

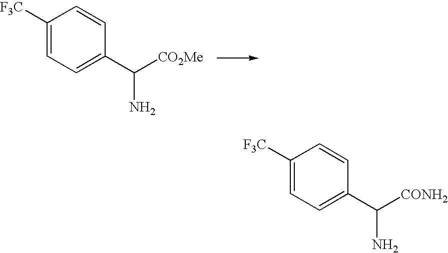

Methyl amino[4-(trifluoromethyl)phenyl]acetate hydrochloride D1 (12 g; 44.5 mmol) was dissolved in 0.88 ammonia (220 ml; ca. 3.3 mol). After stirring overnight at room temperature the reaction mixture was extracted with DCM (150 ml×5) and the extracts dried with Na₂SO₄, filtered, and the solvent evaporated under reduced pressure to afford the title compound (8.92 g; 92%) ¹H NMR (CDCl₃) δ: 1.87 (2H, br s), 4.62 (1H, s), 5.48 (1H, br s), 7.00 (1H, br s) 7.57 (2H, d, J=8 Hz), 7.63 (2H, d, J=8 Hz). Mass Spectrum (Electrospray LC/MS) Found 219 (MH⁺). C₉H₉F₃N₂O requires 218. Ret. time 1.13 min.

### Description 3

### 3-[4-(Trifluoromethyl)phenyl]-1,4-diazaspiro[4.5]decan-2-one

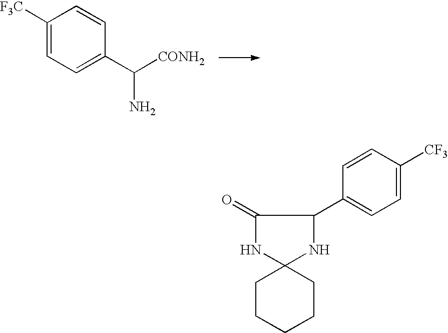

To 2-amino-2-[4-(trifluoromethyl)phenyl]acetamide D2 (8.92 g; 40.9 mmol), in methanol (350 ml) was added cyclohexanone (4.24 ml; 40.9 mmol) and H—Y zeolites (8.92 g) under argon and the mixture refluxed overnight. After cooling to room temperature and chilling in an ice-bath the reaction mixture was filtered. The solid was washed with methanol and the filtrate evaporated under reduced pressure to afford the title compound (10.59 g; 86%) ¹H NMR (CDCl₃) δ: 1.35-1.60 (4H, m), 1.62-1.80 (6H, m), 2.31 (1H, br s), 4.79 (1H, br s) 6.41 (1H, br s), 7.62 (2H, d, J=8 Hz), 7.70 (2H, d, J=8 Hz). Mass Spectrum (Electrospray LC/MS) Found 299 (MH⁺). C₁₅H₁₇F₃N₂O requires 298. Ret. time 2.57 min.

### Description 4

### 3-[4-(Trifluoromethyl)phenyl]-1,4-diazaspiro[4.5]dec-3-en-2-one

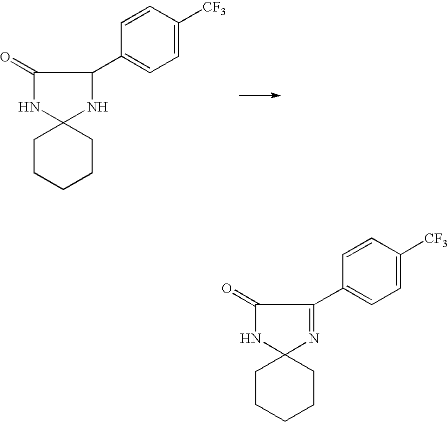

N-Bromosuccinimide (6.32 g; 35.5 mmol; 1 eq) was added to 3-[4-(trifluoromethyl)phenyl]-1,4-diazaspiro[4.5]decan-2-one D3 (10.59 g; 35.5 mmol) in DCM (200 ml) and the reaction stirred overnight at room temperature under argon. Saturated aqueous sodium bicarbonate (150 ml) was added and the mixture stirred, the organic layer was then separated and the aqueous extracted with DCM. The combined DCM extracts were dried with Na₂SO₄, filtered and evaporated under reduced pressure to afford the title product (5 g). Additional washing of the filtered Na₂SO₄ with methanol-DCM several times afforded further title product, giving 10.69 g in total. Mass Spectrum (Electrospray LC/MS) Found 297 (MH⁺). C₁₅H₁₅F₃N₂O requires 296. Ret. time 3.14 min.

### Description 5

### Ethyl {2-oxo-3-[4-(trifluoromethyl)phenyl]-1,4-diazaspiro[4.5]dec-3-en-1-yl}acetate

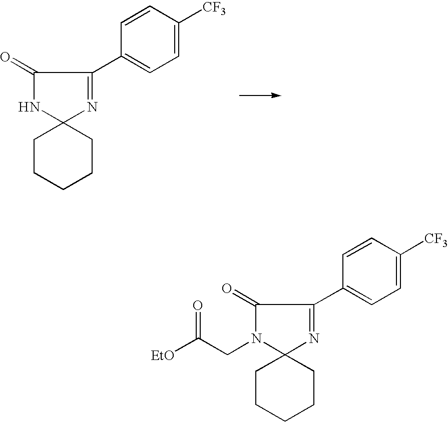

Sodium hydride (0.24 g of a 60% dispersion in oil; 5.95 mmol) was added portionwise over 15 minutes to a stirred solution of 3-[4-(trifluoromethyl)phenyl]-1,4-diazaspiro[4.5]dec-3-en-2-one D4 (1.6 g; 5.41 mmol) in anhydrous DMF (30 ml) at room temperature under an argon atmosphere. On complete addition, the solution was stirred for a further 15 minutes and ethyl bromoacetate (0.99 g; 0.66 ml; 5.95 mmol) was added as a steady stream over 1 minute and the mixture stirred for a further 18 h. Excess sodium hydride was decomposed by careful addition of water, then the resultant mixture poured into water (1 L) and the mixture extracted with diethyl ether (300 ml×4). Combined extracts were washed with water (500 ml), brine (200 ml) then dried (Na₂SO₄) and the solvent evaporated under reduced pressure. The crude product was purified by chromatography on silica gel eluting with 0-50% ethyl acetate in pentane gradient to afford the title compound as a pale yellow waxy solid (1.6 g; 77%). ¹H NMR (CDCl₃) δ: 1.20-1.40 (4H, m, including t, J=7.2 Hz), 1.44-1.49 (1H, m), 1.70-2.10 (8H, m), 4.17 (2H, s), 4.23 (2H, q, J=7.2 Hz), 7.71 (2H, d, J=8 Hz), 8.61 (2H, d, J=8 Hz). Mass Spectrum (Electrospray LC/MS) Found 383 (MH⁺). C₁₉H₂₁F₃N₂O₃ requires 382. Ret. time 3.76 min.

### Description 6

### {2-Oxo-3-[4-(trifluoromethyl)phenyl]-1,4-diazaspiro[4.5]dec-3-en-1-yl}acetic acid

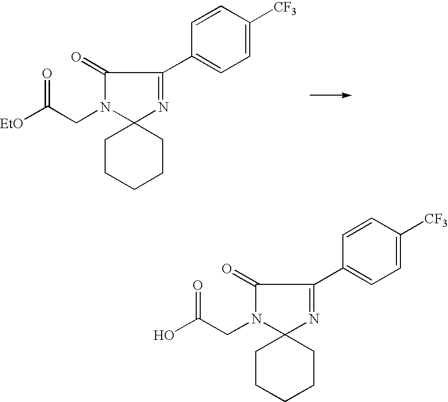

2N Sodium hydroxide (3.18 ml; 6.37 mmol, 1.2 eq) was added to ethyl {2-oxo-3-[4-(trifluoromethyl)phenyl]-1,4-diazaspiro[4.5]dec-3-en-1-yl}acetate D5 (2.03 g; 5.31 mmol) in methanol (70 ml) and water (30 ml) and the mixture stirred at room temperature overnight. The mixture was evaporated in vacuo and the residue partitioned between water and ethyl acetate. The aqueous layer was acidified to pH1 with 5N HCl and extracted with DCM (×3). The organic extracts were passed through a phase separation cartridge and the solvent removed in vacuo to yield the title compound (1.72 g; 91%). Mass Spectrum (Electrospray LC/MS) Found 355 (MH⁺). C₁₇H₁₇F₃N₂O₃ requires 354. Ret. time 3.12 min.

### Description 7

### {2-Oxo-3-[4-(trifluoromethyl)phenyl]-1,4-diazaspiro[4.5]dec-3-en-1-yl}acetyl chloride

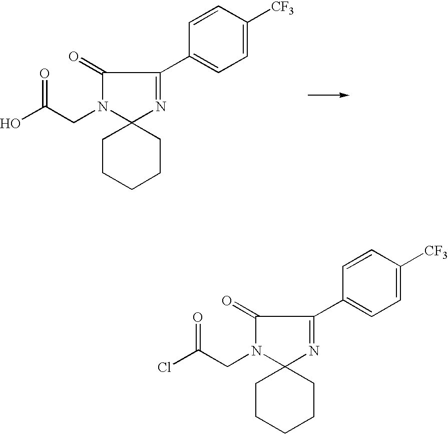

To {2-oxo-3-[4-(trifluoromethyl)phenyl]-1,4-diazaspiro[4.5]dec-3-en-1-yl}acetic acid D6 (1.4 g; 3.95 mmol) in DCM (65 ml) was added oxalyl chloride (0.76 ml; 8.69 mmol; 2.2 eq) and DMF (3 drops) and the reaction was stirred under argon for 1 h at room temperature. Evaporation under reduced pressure afforded the title compound (1.46 g; 99%) which was used without further purification. ¹H NMR (CDCl₃) δ: 1.20-1.55 (3H, m), 1.70-2.15 (7H, m), 4.58 (2H, s), 7.72 (2H, d, J=8 Hz), 8.58 (2H, d, J=8 Hz). Mass Spectrum (Electrospray LC/MS; MeOH) Found 369 (MH⁺ for methyl ester). C₁₈H₁₉F₃N₂O₃ requires 368. Ret. time 3.53 min.

### Description 8

### Methyl amino{4-[(trifluoromethyl)oxy]phenyl}acetate hydrochloride

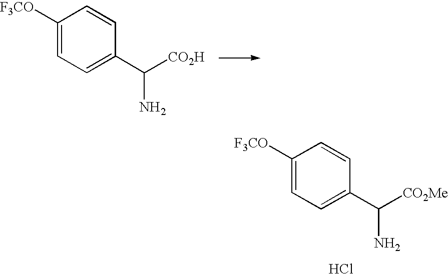

To ice-chilled methanol (30 ml) under argon was carefully added dropwise thionyl chloride (15.44 ml; 0.422 mol) over 30 min. Amino{4-[(trifluoromethyl)oxy]phenyl}acetic acid (5.0 g; 21.280 mmol) was added, ice cooling removed and the reaction mixture was stirred at room temperature for 16 h. The reaction was then evaporated under reduced pressure. Trituration with diethyl ether, followed by filtration provided the title product as the hydrochloride salt, (5.75 g; 95%). ¹H NMR (d₆-DMSO) δ: 3.74 (3H, s), 5.41 (1H, s), 7.51 (2H, d), 7.66 (2H, d), 9.10 (3H, s). Mass Spectrum (Electrospray LC/MS): Found 250 (MH⁺). C₁₀H₁₀F₃NO₃ requires 249. Ret. time 1.52 min.

### Description 9

### 2-Amino-2-{4-[(trifluoromethyl)oxy]phenyl}acetamide

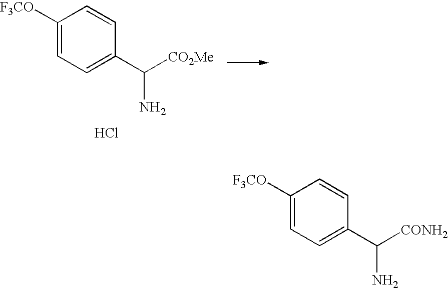

Methyl amino{4-[(trifluoromethyl)oxy]phenyl}acetate D8 as the hydrochloride salt (5.75 g; 20.14 mMol) was dissolved in 0.88 ammonia (75 ml; ca. 1.1 mol) and stirred at room temperature under argon for 16 h. The reaction mixture was extracted with DCM, the extracts dried (MgSO₄) and evaporated under reduced pressure to a white solid, which was dried under reduced pressure to afford the title product (3.70 g; 79%). ¹H NMR (d₆-DMSO) δ: 2.22 (2H, br s), 4.32 (1H, s), 7.08 (1H, br s), 7.30 (2H, d), 7.50 (3H, d). Mass Spectrum (Electrospray LC/MS): Found 235 (MH⁺). C₉H₉F₃N₂O₂ requires 234. Ret. time 1.20 min.

### Description 10

### 3-{4-[(Trifluoromethyl)oxy]phenyl}-1,4-diazaspiro[4.5]decan-2-one

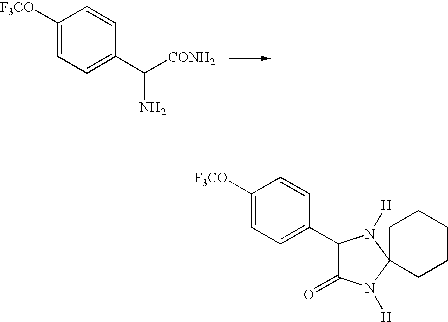

To 2-amino-2-{4-[(trifluoromethyl)oxy]phenyl}acetamide D9 (3.70 g; 15.81 mmol) in methanol (200 ml) was added cyclohexanone (1.549 ml; 15.81 mmol) and H—Y zeolites (6.00 g) and the mixture stirred under reflux for 24 h under argon. The reaction was allowed to cool to room temperature and was filtered and the solid washed well with methanol. The filtrate was evaporated to afford the title product (3.88 g; 50%) as a white solid, after trituration with hexane. ¹H NMR (d₆-DMSO) δ: 1.22-1.45 (2H, m), 1.50-1.70 (8H, m), 3.53 (1H, d), 4.64 (1H, d), 7.32 (2H, d), 7.60 (2H, d), 8.68 (1H, s). Mass Spectrum (Electrospray LC/MS): Found 315 (MH⁺). C₁₅H₁₇F₃N₂O₂ requires 314. Ret. time 2.57 min.

### Description 11

### 3-{4-[(Trifluoromethyl)oxy]phenyl}-1,4-diazaspiro[4.5]dec-3-en-2-one

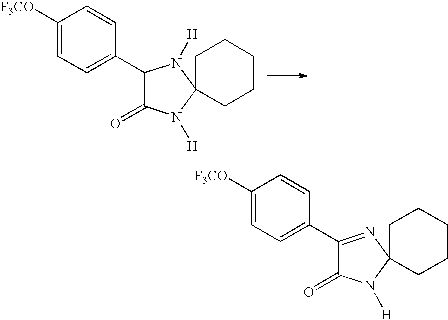

3-{4-[(Trifluoromethyl)oxy]phenyl}-1,4-diazaspiro[4.5]decan-2-one D10 (3.880 g; 12.36 mmol) was dissolved in DCM (80 ml) and stirred at room temperature for 16 hours under an atmosphere of argon with N-bromosuccinimide (2.216 g; 12.36 mmol). A solution of saturated sodium hydrogen carbonate (100 ml) was then added and stirring continued for 1 hour at room temperature. The organic layer was separated, dried (MgSO₄) and evaporated at reduced pressure to yield the title compound as a yellow solid after trituration with hexane (3.25 g; 84%). ¹H NMR (d₆-DMSO) δ: 1.40-1.85 (10H, m), 7.50 (2H, d), 8.47 (2H, d), 10.30 (1H, s). Mass Spectrum (Electrospray LC/MS): Found 313 (MH⁺). C₁₅H₁₅F₃N₂O₂ requires 312. Ret. time 3.23 min.

### Description 12

### Ethyl (2-oxo-3-{4-[(trifluoromethyl)oxy]phenyl}-1,4-diazaspiro[4.5]dec-3-en-1-yl)acetate

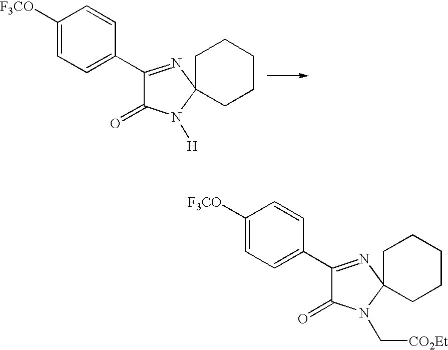

A mixture of 3-{4-[(trifluoromethyl)oxy]phenyl}-1,4-diazaspiro[4.5]dec-3-en-2-one D11 (1.00 g; 3.205 mmol), ethyl bromoacetate (0.354 ml; 3.205 mmol) and potassium carbonate (1.04 g; 7.530 mmol) in DMF (20 ml) was heated at 60° C. for 18 h, with rapid stirring under an atmosphere of argon. After cooling, the reaction solution was poured into water and extracted with ethyl acetate. The organic layer was separated, washed with saturated brine, dried (MgSO₄) and evaporated. The residue was chromatographed over silica gel (50 g), eluting with ethyl acetate-pentane mixtures to afford the title product (0.625 g; 50%) as a colourless oil. ¹H NMR (CDCl₃) δ: 1.24-1.38 (4H, m), 1.41-1.50 (2H, m), 1.74-2.11 (7H, m), 4.18 (2H, s), 4.27 (2H, q), 7.30 (2H, d), 8.57 (2H, d). Mass Spectrum (Electrospray LC/MS): Found 399 (MH⁺). C₁₉H₂₁F₃N₂O₄ requires 398. Ret. time 3.77 min.

### Description 13

### (2-Oxo-3-{4-[(trifluoromethyl)oxy]phenyl}-1,4-diazaspiro[4.5]dec-3-en-1-yl)acetic acid

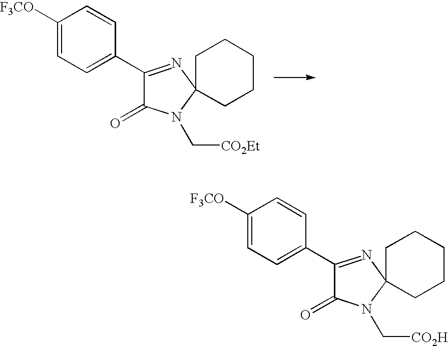

To a stirred mixture of ethyl (2-oxo-3-{4-[(trifluoromethyl)oxy]phenyl}-1,4-diazaspiro[4.5]dec-3-en-1-yl)acetate D12 (0.625 g; 1.57 mmol) in water (30 ml) and methanol (10 ml) was added 2N sodium hydroxide solution (0.95 ml; 1.88 mmol). The reaction solution was heated at 60° C. for 16 hours, cooled and evaporated under reduced pressure. The residue was partitioned between water and ethyl acetate. The aqueous layer was acidified with 5N HCl and extracted into DCM. The DCM extracts were dried (MgSO₄) and evaporated under reduced pressure to afford the title acid (0.513 g; 89%) as a white solid. ¹H NMR (d₆-DMSO) δ: 1.22-1.42 (3H, m), 1.70-1.95 (5H, m), 1.98-2.09 (2H, m), 4.21 (2H, s), 7.58 (2H, d), 8.50 (2H, d), 12.90 (1H, broad s). Mass Spectrum (Electrospray LC/MS): Found 371 (MH⁺). C₁₇H₁₇F₃N₂O₄ requires 370. Ret. time 3.22 min.

### Description 14

### (2-Oxo-3-{4-[(trifluoromethyl)oxy]phenyl}-1,4-diazaspiro[4.5]dec-3-en-1-yl)acetyl chloride

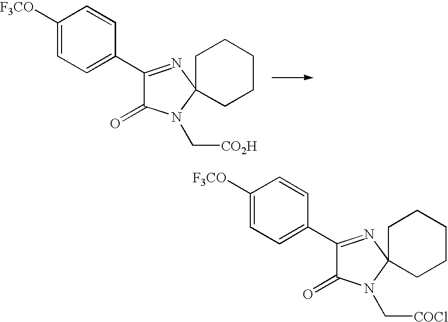

To a suspension of (2-oxo-3-{4-[(trifluoromethyl)oxy]phenyl}-1,4-diazaspiro[4.5]dec-3-en-1-yl)acetic acid D13 (375 mg; 1.014 mmol) in DCM (15 ml) was added oxalyl chloride (0.205 ml; 2.028 mmol) followed with stirring by DMF (1 drop). After stirring overnight the reaction was evaporated under reduced pressure to afford the title product (396 mg; 100%) as a pale yellow solid which was used without further purification. Mass Spectrum (Electrospray LC/MS; MeOH): Found 385 (MH⁺ for methyl ester). C₁₈H₁₉F₃N₂O₄ requires 384. Ret. time 3.62 min.

### Description 15

### Methyl amino(3-chlorophenyl)acetate

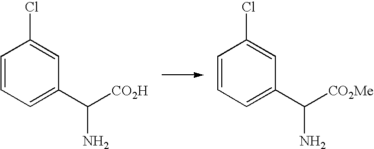

To amino(3-chlorophenyl)acetic acid (5.0 g; 26.95 mmol) suspended in methanol (30 ml) and stirred at ice-bath temperature under argon was added dropwise thionyl chloride (30 ml) over 30 min. After stirring for a further 2 h at 5° C. and at room temperature for 16 h, the reaction solution was evaporated under reduced pressure. Trituration of the residue with diethylether afforded the title compound as a ca. 1:1 mixture with amino(3-chlorophenyl)acetic acid (6.16 g).

### Description 16

### 2-Amino-2-(3-chlorophenyl)acetamide

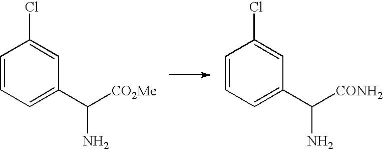

Methyl amino(3-chlorophenyl)acetate D15 (6.16 g) was dissolved in concentrated ammonia solution (75 ml) and stirred at room temperature for 16 h. The reaction was extracted with DCM (×2) and the extracts dried, evaporated and triturated with hexane to afford the title compound (1.527 g; 31% from amino(3-chlorophenyl)acetic acid). ¹H NMR (d₆-DMSO) δ: 2.22 (2H, br s), 4.31 (1H, s), 7.09 (1H, br s), 7.28-7.39 (3H, m), 7.48 (1H, s), 7.51 (1H, br s).

### Description 17

### 3-(3-Chlorophenyl)-1,4-diazaspiro[4.5]decan-2-one

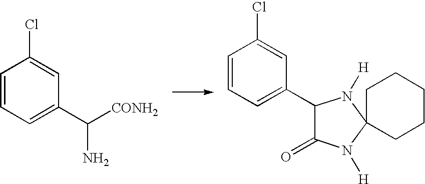

The title compound (1.49 g; 68%) was prepared from 2-amino-2-(3-chlorophenyl)acetamide D16 (1.52 g; 8.26 mmol) and cyclohexanone (0.810 g; 8.26 mmol) in methanol (100 ml) with H—Y zeolites (3.5 g) in a similar manner to the procedure of D10. ¹H NMR (d₆-DMSO) δ: 1.22-1.43 (2H, m), 1.48-1.70 (8H, m), 3.58 (1H, d), 4.60 (1H, d), 7.29-7.40 (2H, m), 7.47 (1H, d), 7.51 (1H, s), 8.68 (1H, s).

### Description 18

### 3-(3-Chlorophenyl)-1,4-diazaspiro[4.5]dec-3-en-2-one

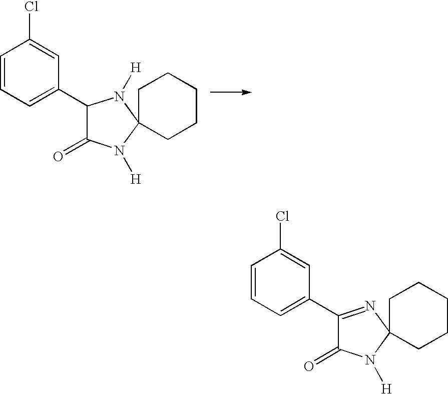

The title compound (1.363 g, 91%) was prepared from 3-(3-chlorophenyl)-1,4-diazaspiro[4.5]decan-2-one D17 (1.49 g; 5.64 mmol) and N-bromosuccinimide (1.011 g; 5.64 mmol) in DCM (30 ml) by a similar procedure to that described in D11. ¹H NMR (d₆-DMSO) δ: 1.40-1.88 (10H, m), 7.58 (1H, t), 7.64 (1H, m), 8.28 (1H, d), 8.37 (1H, s), 10.34 (1H, br s).

### Description 19

### Ethyl[3-(3-chlorophenyl)-2-oxo-1,4-diazaspiro[4.5]dec-3-en-1-yl]acetate

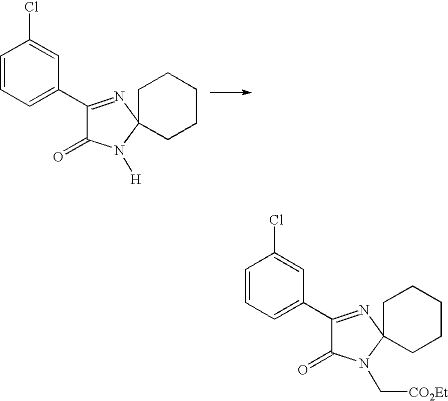

The title compound (0.876 g, 76%) was prepared from 3-(3-chlorophenyl)-1,4-diazaspiro[4.5]dec-3-en-2-one D18 (0.870 g, 3.32 mmol), potassium carbonate (1.078 g; 7.80 mmol), and ethyl bromoacetate (0.553 g; 3.31 mmol) in DMF (20 ml) by a similar procedure to that described in D12. ¹H NMR (CDCl₃) δ: 1.30 (3H, t), 1.42-1.50 (2H, m), 1.73-2.13 (8H, m), 4.18 (2H, s), 4.22 (2H, q), 7.40 (1H, t), 7.48 (1H, m), 8.40-8.49 (2H, m).

### Description 20

### [3-(3-Chlorophenyl)-2-oxo-1,4-diazaspiro[4.5]dec-3-en-1-yl]acetic acid

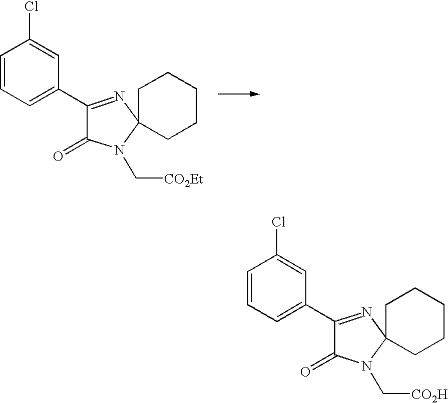

The title compound (0.520 g, 65%) was prepared from ethyl[3-(3-chlorophenyl)-2-oxo-1,4-diazaspiro[4.5]dec-3-en-1-yl]acetate D19 (0.876 g, 2.517 mmol) by reaction with 2N sodium hydroxide solution (1.51 ml) in methanol (10 ml) and water (30 ml) by a similar procedure to that described in D13. ¹H NMR (CDCl₃) δ: 1.25-1.39 (1H, m), 1.41-1.51 (2H, br d), 1.78-2.11 (7H, m), 4.21 (2H, s), 7.40 (1H, t), 7.48 (1H, m), 8.38-8.47 (2H, m).

### Description 21

### [3-(3-Chlorophenyl)-2-oxo-1,4-diazaspiro[4.5]dec-3-en-1-yl]acetyl chloride

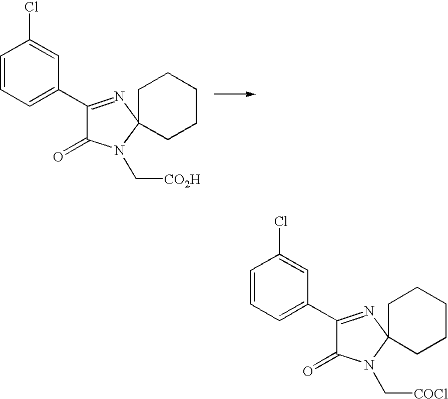

The title compound (0.420 g, 99%) was prepared from [3-(3-chlorophenyl)-2-oxo-1,4-diazaspiro[4.5]dec-3-en-1-yl]acetic acid D20 (0.400 g, 1.25 mmol), oxalyl chloride (0.253 ml; 2.90 mmol) and DMF (1 drop) in DCM (15 ml) by a similar procedure to that described in D14 and was used without further purification.

### Description 22

### 2-Amino-2-(4-bromophenyl)acetamide

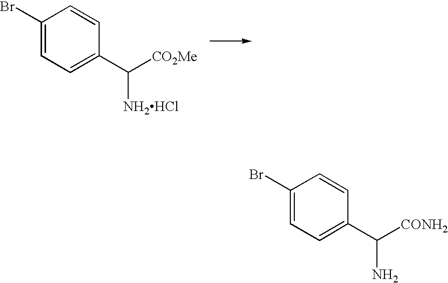

Methyl amino(4-bromophenyl)acetate hydrochloride (commercially available from Bionet Research) (5.0 g; 17.822 mmol) was elaborated to the title compound (2.69 g; 66%) using concentrated ammonia solution (75 ml) using a similar procedure to that described in D9. ¹H NMR (d₆-DMSO) δ: 2.20 (2H, br s), 4.38 (1H, s), 7.08 (1H, br s), 7.36 (2H, d), 7.50 (3H, d).

### Alternative Procedure

Methyl amino(4-bromophenyl)acetate hydrochloride (commercially available from Bionet Research) (9.6 g) was elaborated to the title compound (6.4 g; 81%) using 0.880 ammonia solution (300 ml) using a similar procedure to that described in D9. ¹H NMR (d₆-DMSO) δ: 2.20 (2H, br s), 4.38 (1H, s), 7.08 (1H, br s), 7.36 (2H, d), 7.50 (3H, d).

### Description 23

### 3-(4-Bromophenyl)-1,4-diazaspiro[4.5]decan-2-one

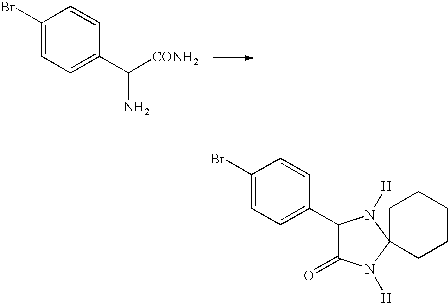

The title compound (2.22 g; 61%) was prepared from 2-amino-2-(4-bromophenyl)acetamide D22 (2.69 g; 11.75 mmol), cyclohexanone (1.22 ml; 11.75 mmol; 1 eq) and H—Y zeolites (2.69 g) in methanol (100 ml) using a similar procedure to that described in D10. ¹H NMR (d₆-DMSO) δ: 1.22-1.43 (2H, m), 1.48-1.70 (8H, m), 3.50 (1H, d), 4.58 (1H, d), 7.43 (2H, d), 7.51 (2H, d), 8.62 (1H, s).

### Description 24

### 3-(4-Bromophenyl)-1,4-diazaspiro[4.5]dec-3-en-2-one

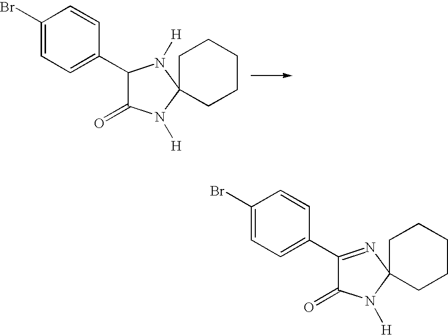

The title compound was prepared from 3-(4-bromophenyl)-1,4-diazaspiro[4.5]decan-2-one D23 (4.96 g) and N-bromosuccinimide (2.88 g; 1 eq) in DCM (100 ml) using a similar procedure to that described in D11. Yield 1.69 g. ¹H NMR (d₆-DMSO) δ: 1.42-1.88 (10H, m), 7.70 (2H, d), 8.28 (2H, d), 10.30 (1H, br s).

### Description 25

### 2-Chloro-N-(3,4-difluorophenyl)acetamide

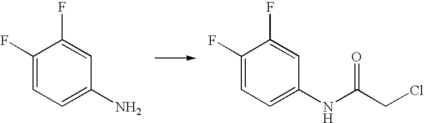

A mixture of 3,4-difluoroaniline (commercially available; 6.46 g; 50 mmol; 5 ml) and chloroacetyl chloride (5.65 g; 50 mmol; 4 ml) in dioxan (50 ml) was heated with stirring for 1 h. The solution was concentrated to 25 ml, cooled to room temperature and some water added. The resulting precipitate was filtered off and dried to afford the title product. The product was further dried overnight in an oven (9.41 g; 91.5%). ¹H NMR (CDCl₃) δ: 4.20 (2H, s), 7.13-7.16 (2H, m), 7.63-7.68 (1H, s), 8.23 (1H, br s).

### Description 26

### 2-Chloro-N-(2,4-dimethylphenyl)acetamide

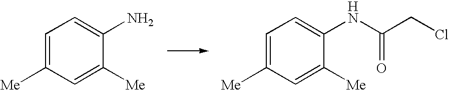

Chloroacetyl chloride (4.00 ml, 50.0 mmol) was slowly added to 2,4-dimethylaniline (6.05 g, 50.0 mmol) in dioxan (50 ml) stirred at room temperature. The mixture was then heated to reflux for 1 hour. The solution was cooled and water (50 ml) added. The resulting precipitate was filtered and dried to afford the desired product as a pale solid (8.03 g, 81%). ¹H NMR (CDCl₃) δ 2.26 (3H, s), 2.31 (3H, s), 4.23 (2H, s), 7.03 (1H, s), 7.04 (1H, d, J=8.4 Hz), 7.68 (1H, d, J=8.4 Hz), 8.15 (1H, broad s). Mass Spectrum (Electrospray LC/MS): Found 198 (MH⁺). C₁₀H₁₂³⁵CINO requires 197. Ret. time 2.43 min.

### Description 27

### Methyl({[(1,1-dimethylethyl)oxy]carbonyl}amino)(4-hydroxyphenyl)acetate

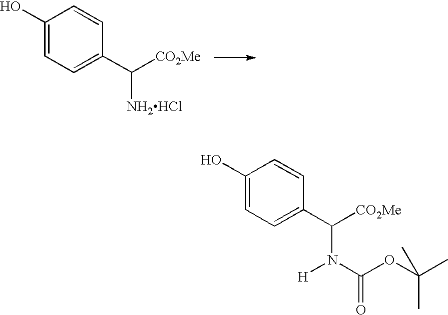

Methyl (2S)-amino(4-hydroxyphenyl)ethanoate hydrochloride (commercially available from Sigma Aldrich; 3.0 g; 13.80 mmol) was dissolved in DCM (100 ml). Di-tert-butyl dicarbonate (3.16 g; 14.48 mmol) was added to this solution, followed by triethylamine (4.22 ml; 30.50 mmol). The resulting solution was stirred at room temperature for 16 h under an atmosphere of argon. The solution was then washed with water, dried (MgSO₄) and evaporated at reduced pressure to yield the title compound as a colourless gum (4.60 g; 100%). ¹H NMR (CDCl₃) δ: 1.42 (9H, s), 3.71 (3H, s), 5.22 (1H, br d), 5.50 (1H, br d), 6.78 (2H, d), 7.20 (2H, d).

### Description 28

### Methyl({[(1,1-dimethylethyl)oxy]carbonyl}amino)(4-{[2-(methyloxy)ethyl]oxy}phenyl)ethanoate

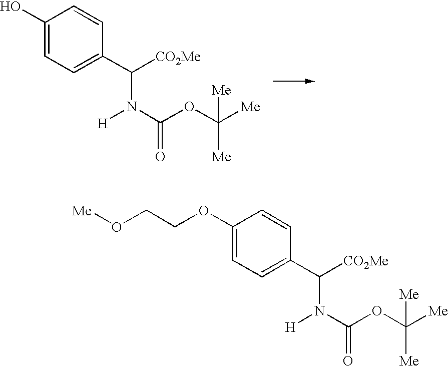

Methyl({[(1,1-dimethylethyl)oxy]carbonyl}amino)(4-hydroxyphenyl)acetate D27 (2.70 g; 9.608 mmol), triphenylphosphine (2.517 g; 9.608 mmol) and 2-methoxyethanol (0.730 g; 9.608 mmol) were dissolved in dry THF (100 ml) and cooled to 5° C. under an atmosphere of argon. A solution of diisopropylazodicarboxylate (2.142 g; 9.608 mmol) in dry THF (20 ml) was added dropwise to the cooled, stirred solution over a period of 15 minutes. The resulting solution was stirred at ambient temperature for 16 h and then partitioned between ethyl acetate and water. The organic solution was washed with saturated brine, dried (MgSO₄) and evaporated at reduced pressure. The residual oil was chromatographed over silica gel (50 g), eluting with a gradient of 0 to 50% ethyl acetate-pentane. The title compound was obtained as a colourless oil (4.1 g; 100%). ¹H NMR (CDCl₃) δ: 1.44 (9H, s), 3.46 (3H, s), 3.71 (3H, s), 3.74 (2H, m), 4.10 (2H, m), 5.26 (1H, br d), 5.49 (1H, br d), 6.90 (2H, d), 7.28 (2H, d).

### Description 29

### Methyl amino(4-{[2-(methyloxy)ethyl]oxy}phenyl)acetate

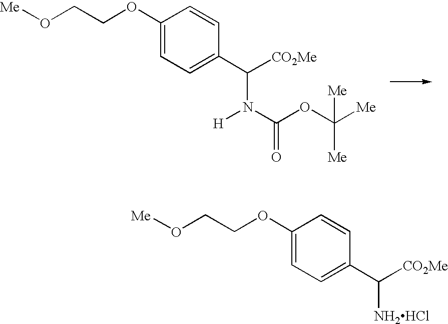

Methyl({[(1,1-dimethylethyl)oxy]carbonyl}amino)(4-{[2-(methyloxy)ethyl]oxy}phenyl)ethanoate D28 (4.1 g; 12.09 mmol) was dissolved in a mixture of DCM (25 ml) and trifluoroacetic acid (25 ml) and the resulting solution was stirred at room temperature under an atmosphere of argon for 16 h. The solution was then evaporated at reduced pressure and the residue was partitioned between ethyl acetate and 2N HCl solution. The aqueous layer was separated and evaporated at reduced pressure to yield the title compound as the hydrochloride salt. Mass Spectrum (Electrospray LC/MS): Found 262 (MNa⁺). C₁₂H₁₇NO₄ requires 239. Ret. time 1.13 min. This material was used without further purification in the next stage.

### Description 30

### 2-Amino-2-(4-{[2-(methyloxy)ethyl]oxy}phenyl)acetamide

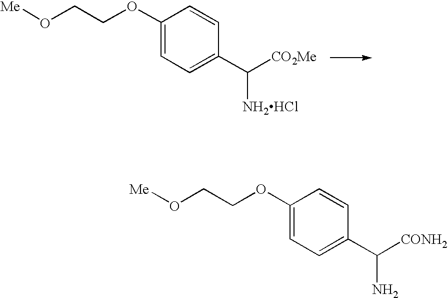

The hydrochloride salt of methyl amino(4-{[2-(methyloxy)ethyl]oxy}phenyl)acetate D29 was dissolved in concentrated 0.88 ammonia solution (100 ml) and allowed to stand at room temperature for 16 h. The solution was then evaporated at reduced pressure. The resulting solid was triturated with hexane and collected by filtration to yield the title compound (2.71 g; 100% yield from D39) as a white solid after drying in vacuo. ¹H NMR inter alia (d₆-DMSO) δ: 3.30 (3H, s), 3.65 (2H, m), 4.09 (2H, m), 4.81 (1H, s), 7.00 (2H, d), 7.46 (2H, d).

### Description 31

### 3-(4-{[(Methyloxy)ethyl]oxy}phenyl)-1,4-diazaspiro[4.5]decan-2-one

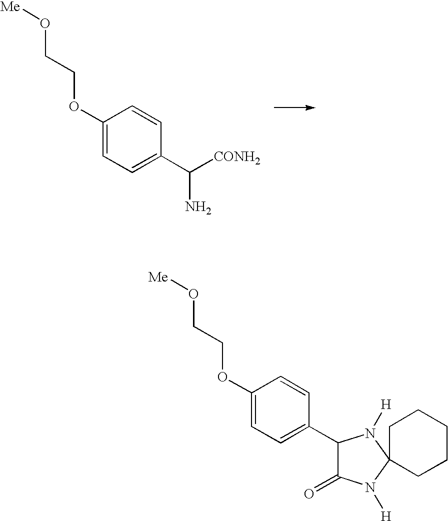

The title compound (0.132 g; 4%) was prepared from 2-amino-2-(4-{[2-(methyloxy)ethyl]oxy}phenyl)acetamide D30 (2.70 g; 12.054 mmol), cyclohexanone (1.275 ml; 12.054 mmol) and H—Y zeolites (5.5 g) in methanol (100 ml) by a similar procedure to that described in D10. Mass Spectrum (Electrospray LC/MS): Found 305 (MH⁺). C₁₇H₂₄N₂O₃ requires 304. Ret. time 1.42 min.

### Description 32

### 3-(4-{[(Methyloxy)ethyl]oxy}phenyl)-1,4-diazaspiro[4.5]dec-3-en-2-one

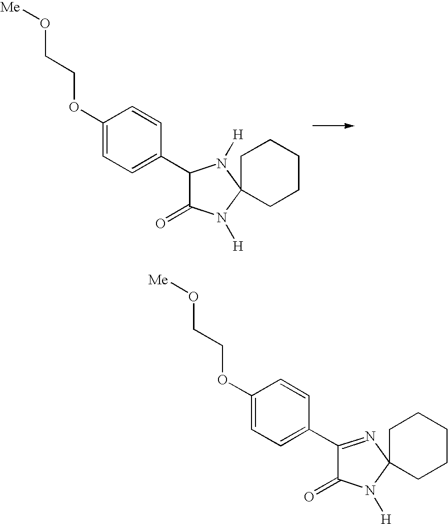

The title compound (0.093 g; 70%) was prepared from 3-(4-{[(methyloxy)ethyl]oxy}phenyl)-1,4-diazaspiro[4.5]decan-2-one D31 (0.132 g; 0.434 mmol) and N-bromosuccinimide (0.078 g; 0.434 mmol) in DCM (20 ml) by a similar procedure to that described in D11. ¹H NMR (CDCl₃) δ: 1.48-1.72 (6H, m), 1.88-2.05 (4H, m), 3.49 (3H, s), 3.79 (2H, m), 4.19 (2H, m), 7.00 (2H, d), 7.92 (1H, br s), 8.40 (2H, d). Mass Spectrum (Electrospray LC/MS): Found 303 (MH⁺). C₁₇H₂₂N₂O₃ requires 302. Ret. time 2.50 min.

### Description 33

### 2-Bromo-N-(3,5-difluorophenyl)acetamide

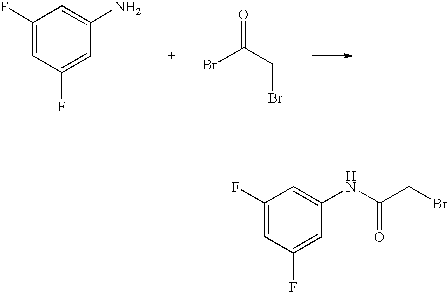

A mixture of 3,5-difluoroaniline (10 g; 77.45 mmol) and bromoacetyl bromide (6.73 ml; 77.45 mmol) in anhydrous dioxan (100 ml) was refluxed for 1.5 h, cooled to room temperature and diluted with water (400 ml) to afford a gum. The mother liquors were decanted and water added (200 ml), followed by ethyl acetate (300 ml). After stirring for 10 min the layers were separated and the organics dried (Na₂SO₄) and evaporated under reduced pressure. Recrystallisation from ethyl acetate-pentane afforded the title product as pale yellow crystals (6.5 g; 33%). ¹H NMR (CDCl₃) δ: 4.02 (2H, s), 6.60-6.65 (1H, m), 7.14-7.20 (2H, m), and 8.16 (1H, br s).

### Description 34

### 3-(4-Bromophenyl)-1,4-diazaspiro[4.4]nonan-2-one

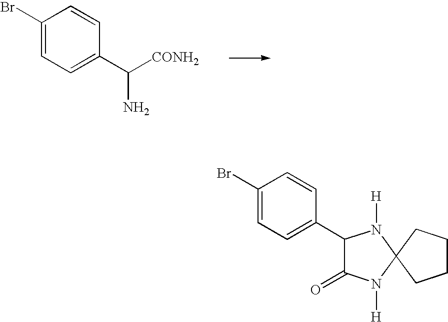

The title compound was prepared from 2-amino-2-(4-bromophenyl)acetamide D22 (2.29 g; 10 mmol), cyclopentanone (0.9 ml; 10 mmol) and H—Y zeolites (3 g) in ethanol (200 ml) using a similar procedure to that described in D10, except that further cyclopentanone (0.9 ml) and H—Y zeolites (3 g) were added after 20 hours of reflux and the heating continued for a further 24 hours. After work-up the title compound (1.91 g; 65%) was obtained as a colourless solid. Mass Spectrum (Electrospray LC/MS) Found 295 (MH⁺). C₁₃H₁₅⁷⁹BrN₂O requires 294. Ret. time 1.83 min.

### Description 35

### 3-(4-Bromophenyl)-1,4-diazaspiro[4.4]non-3-en-2-one

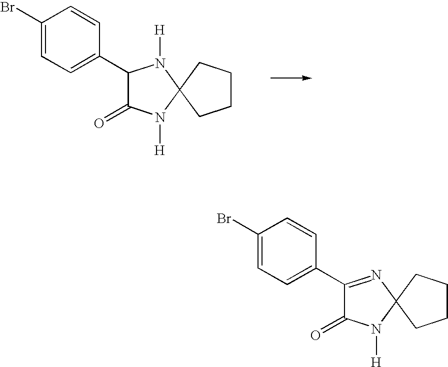

The title compound (1.80 g; 94%) was prepared from 3-(4-bromophenyl)-1,4-diazaspiro[4.4]nonan-2-one D34 (1.91 g; 6.48 mmol) and N-bromosuccinimide (1.153 g; 6.48 mmol) in DCM (150 ml) using a similar procedure to that described in D11, except that the initial reaction mixture was stirred for 66 hours instead of 16 hours and the amount of saturated sodium hydrogen carbonate used was 300 ml and the mixture was stirred for a further 2 hours following the addition of the sodium hydrogen carbonate. Mass Spectrum (Electrospray LC/MS) Found 293 (MH⁺). C₁₃H₁₃⁷⁹BrN₂O requires 292. Ret. time 2.73 min.

### Description 36

### 4-(3-Oxo-1,4-diazaspiro[4.4]non-1-en-2-yl)benzonitrile

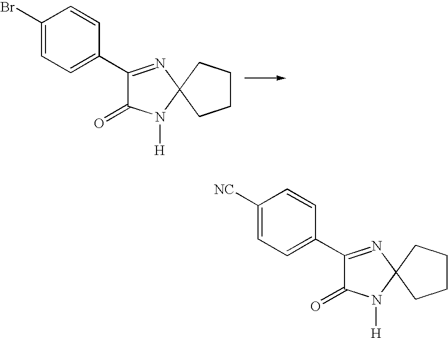

Copper (I) cyanide (0.92 g; 10.24 mmol) was added in one portion to a rapidly stirred mixture of 3-(4-bromophenyl)-1,4-diazaspiro[4.4]non-3-en-2-one D35 (1.5 g; 5.12 mmol) in NMP (25 ml) under an argon atmosphere and was heated at vigorous reflux for 3 hours. On cooling water (0.5 L) and ethyl acetate (300 ml) were added and the mixture filtered through kieselguhr. The filtrate layers were separated and the aqueous layer extracted with ethyl acetate (300 ml). Combined organics were dried (Na₂SO₄) and evaporated under reduced pressure. Chromatography on silica gel eluting with 0-100% ethyl acetate in pentane gradient gave the title compound as a pale orange solid (490 mg; 40%). Mass Spectrum (Electrospray LC/MS) Found 240 (MH⁺). C₁₄H₁₃N₃O requires 239. Ret. time 2.42 min.

### Description 37

### 4-(3-Oxo-1,4-diazaspiro[4.5]dec-1-en-2-yl)benzonitrile

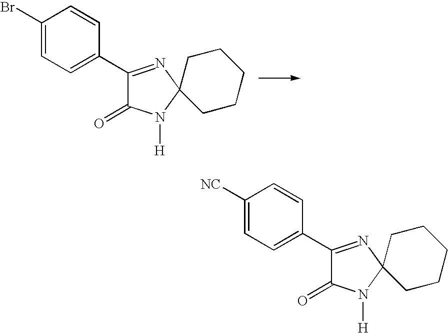

The title compound (420 mg) was prepared from 3-(4-bromophenyl)-1,4-diazaspiro[4.5]dec-3-en-2-one D24 (1.0 g; 3.26 mmol) and copper (I) cyanide (587 mg; 2 eq) in NMP (20 ml) using a similar procedure to that described in D36, except that the product was crystallised from diethyl ether/hexane to give a white solid (420 mg). From the mother liquors, an additional quantity of the title compound (0.321 mg) was obtained. Mass Spectrum (Electrospray LC/MS) Found 254 (MH⁺). C₁₅H₁₅N₃O requires 253. Ret. time 2.64 min. A further quantity of the title compound (0.406 g; 29%) was isolated from the top of the chromatography column.

### Description 38

### Ethyl[3-(4-cyanophenyl)-2-oxo-1,4-diazaspiro[4.5]dec-3-en-1-yl]acetate

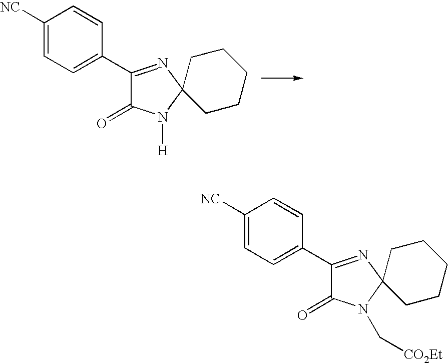

The title compound (370 mg; 86%) was prepared from 4-(3-oxo-1,4-diazaspiro[4.5]dec-1-en-2-yl)benzonitrile D37 (321 mg; 1.27 mmol), ethyl bromoacetate (0.281 ml; 2.54 mmol; 2 eq) and potassium carbonate (350 mg; 2.54 mmol) in DMF (10 ml) by a similar procedure to that described in D12, except that the initial heating was for 3 days and the product was obtained directly from evaporation of the organic phase after work-up. Mass Spectrum (Electrospray LC/MS) Found 340 (MH⁺). C₁₉H₂₁N₃O₃ requires 339. Ret. time 3.21 min.

### Description 39

### [3-(4-Cyanophenyl)-2-oxo-1,4-diazaspiro[4.5]dec-3-en-1-yl]acetic acid

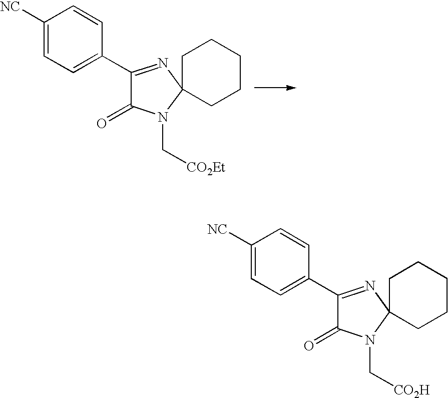

A solution of ethyl[3-(4-cyanophenyl)-2-oxo-1,4-diazaspiro[4.5]dec-3-en-1-yl]acetate D38 (0.37 g; 1.09 mmol) and 2N sodium hydroxide (1.09 ml; 2 eq) in 50% aqueous methanol (20 ml) was heated under argon at 60° C. for 16 hours, cooled and evaporated to a small volume under reduced pressure. Water was added, the mixture acidified to pH1 with 5M HCl and extracted twice with DCM. Combined organics were washed with brine, dried and evaporated to give the title compound as a pale yellow solid (196 mg; 57%). Mass Spectrum (Electrospray LC/MS) Found 312 (MH⁺). C₁₇H₁₇N₃O₃ requires 311. Ret. time 2.58 min.

### Description 40

### [3-(4-Cyanophenyl)-2-oxo-1,4-diazaspiro[4.5]dec-3-en-1-yl]acetyl chloride

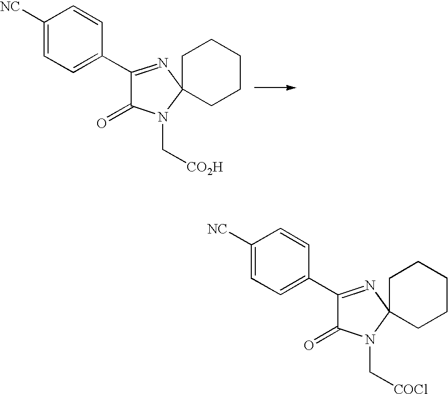

The title compound (0.201 g; 96%) was prepared from [3-(4-cyanophenyl)-2-oxo-1,4-diazaspiro[4.5]dec-3-en-1-yl]acetic acid D39 (196 mg; 0.63 mmol), oxalyl chloride (0.106 ml; 1.26 mmol; 2 eq) and DMF (1 drop) in DCM (20 ml) by a similar procedure to that described in D14 and was used without further purification.

### Description 41

### 3-[4-(Trifluoromethyl)phenyl]-1,4-diazaspiro[4.4]nonan-2-one

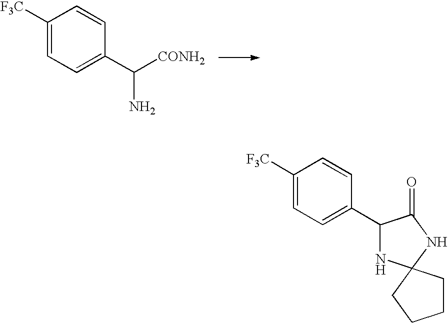

Cyclopentanone (3.32 ml, 37.6 mmol) was added to a stirred solution of 2-amino-2-[4-(trifluoromethyl)phenyl]acetamide D2 (8.2 g, 37.6 mmol) in methanol (80 ml). p-Toluenesulfonic acid monohydrate (71 mg, 0.38 mmol) was then added and the mixture left to stir under reflux for 18.5 h. The mixture was concentrated in vacuo and the solid residue partitioned between DCM and a dilute aqueous solution of sodium bicarbonate (1:4 saturated aqueous sodium bicarbonate solution:water). The layers were separated and the aqueous extracted twice with DCM. The DCM extracts were combined, dried (Na₂SO₄), filtered and concentrated in vacuo to give the title compound as a cream amorphous solid (9.89 g, 92%); ¹H NMR (d₆-DMSO) δ: 1.60-1.85 (7H, m), 2.25-2.90 (1H, m), 3.72 (1H, d), 4.66 (1H, d), 7.60-7.75 (4H, m), 8.60 (1H, br s).

### Description 42

### 3-[4-(Trifluoromethyl)phenyl]-1,4-diazaspiro[4.4]non-3-en-2-one

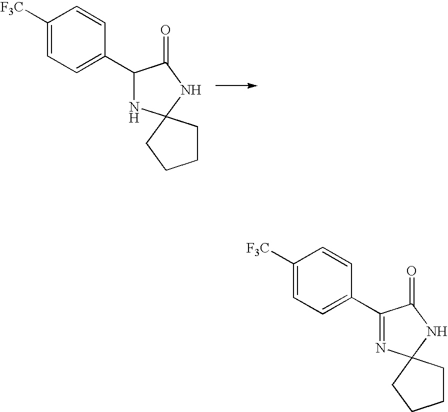

N-Bromosuccinimide (6.69 g, 37.6 mmol) was added to a stirred solution of 3-[4-(trifluoromethyl)phenyl]-1,4-diazaspiro[4.4]nonan-2-one D41 (9.89 g, 34.8 mmol) in DCM (320 ml). The mixture was left to stir at room temperature for 17.5 h. Saturated aqueous sodium bicarbonate solution (ca. 300 ml) was added and the mixture stirred at room temperature for 30 mins. The DCM layer was then separated and the aqueous extracted twice with DCM. The DCM extracts were combined, dried (MgSO₄), filtered and concentrated in vacuo to give a solid black residue. The residue was dissolved in DCM (300 ml) and saturated aqueous sodium bicarbonate solution (300 ml) was added. The mixture was then stirred vigorously for 18 h. The DCM layer was separated and the aqueous was extracted twice with DCM. The DCM extracts were combined, dried (MgSO₄), filtered and concentrated in vacuo to give a black residue. This residue was purified by silica gel chromatography eluting with 20-60% ethyl acetate/cyclohexane to give the title compound as a cream amorphous solid (2.90 g, 30%); ¹H NMR (CDCl₃) δ: 1.90-2.20 (8H, m), 7.73 (2H, d), 8.53 (2H, d), 8.65 (1H, br s).

### Description 43

### Ethyl {2-oxo-3-[4-(trifluoromethyl)phenyl]-1,4-diazaspiro[4.4]non-3-en-1-yl}acetate

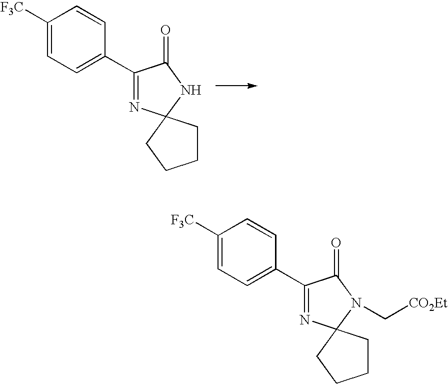

Ethyl bromoacetate (2.75 ml, 24.8 mmol) and then potassium carbonate (754 mg, 5.46 mmol) were added to a stirred solution of 3-[4-(trifluoromethyl)phenyl]-1,4-diazaspiro[4.4]non-3-en-2-one D42 (1.40 g, 4.96 mmol) in acetone (75 ml). The mixture was then stirred under reflux for 47 h. More potassium carbonate (754 mg, 5.46 mmol) was then added and the mixture left to stir for a further 20 h. The mixture was cooled to room temperature and partitioned between DCM and water. The DCM layer was separated and the aqueous extracted twice with DCM. The DCM extracts were combined, dried (MgSO₄), filtered and concentrated in vacuo to give a yellow oil (ca. 6.5 g). Purification by silica gel chromatography eluting with 20-60% ethyl acetate/cyclohexane gave the title compound as a white amorphous solid (1.46 g, 80%); ¹H NMR (CDCl₃) δ: 1.30 (3H, t), 1.82-2.05 (6H, m), 2.10-2.20 (2H, m), 4.19 (2H, s), 4.25 (2H, q), 7.72 (2H, d), 8.58 (2H, d).

### Description 44

### {2-Oxo-3-[4-(trifluoromethyl)phenyl]-1,4-diazaspiro[4.4]non-3-en-1-yl}acetic acid

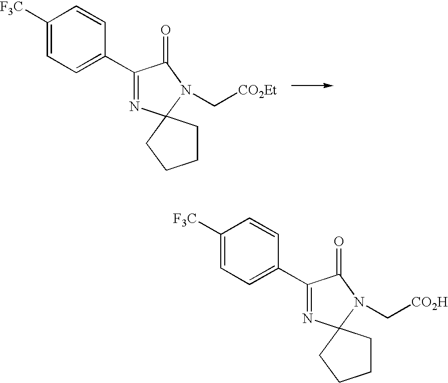

Sodium hydroxide (190 mg, 4.75 mmol) was added to a stirred solution of ethyl {2-oxo-3-[4-(trifluoromethyl)phenyl]-1,4-diazaspiro[4.4]non-3-en-1-yl}acetate D43 (1.46 g, 3.96 mmol) in water (45 ml)/methanol (14 ml). The mixture was then heated at 60° C. for 24 h. More sodium hydroxide (95 mg, 2.38 mmol) was added and the mixture stirred for a further 17 h. Mixture was cooled and then concentrated in vacuo. The residue was partitioned between ethyl acetate and dilute aqueous sodium bicarbonate solution (ca. 1:10 saturated aqueous sodium bicarbonate solution:water). The layers were separated and the ethyl acetate layer was extracted twice with dilute aqueous sodium bicarbonate solution. The aqueous extracts were combined and acidified to pH 2 with 2M HCl. The aqueous was then extracted 3 times with DCM. The DCM extracts were combined, dried (MgSO₄), filtered and concentrated in vacuo to give a white amorphous solid (1.15 g, 85%); ¹H NMR (CDCl₃) δ: 1.80-2.05 (6H, m), 2.10-2.20 (2H, m), 4.25 (2H, s), 7.72 (2H, d), 8.54 (2H, d).

### Description 45

### 2-Amino-2-[4-(methyloxy)phenyl]acetamide

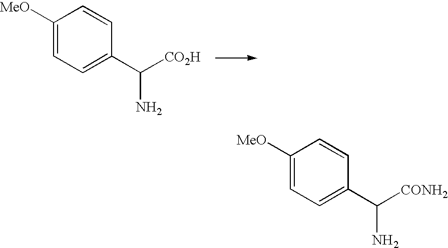

To an ice-cold suspension of 4-methoxyphenylglycine (3.77 g; 0.021 mol) in methanol was added thionyl chloride dropwise over 30 min. After complete addition, the reaction mixture was heated at reflux for 3 h, cooled and evaporated. The resulting solid was dissolved in 0.88 ammonia (100 ml) and stirred at room temperature overnight. The reaction was extracted twice with DCM and the organic phases separated with a Phase-Separation cartridge and evaporated under reduced pressure to afford the title product (0.45 g; 12%) as a white solid. ¹H NMR (CDCl₃) δ: 1.77 (2H, br s), 3.80 (3H, s), 4.50 (1H, s), 5.52 (1H, s), 6.83 (1H, s), 6.87-6.91 (2H, m), 7.33-7.36 (2H, m).

### Description 46

### 3-[4-(Methyloxy)phenyl]-1,4-diazaspiro[4.5]decan-2-one

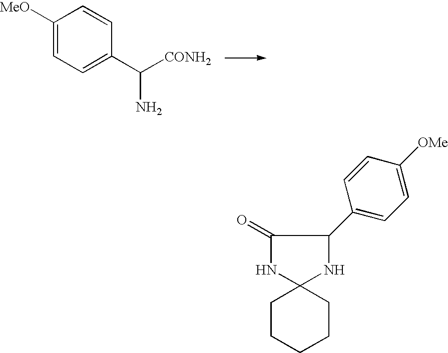

The title compound (0.420 g; 65%) was obtained from 2-amino-2-[4-(methyloxy)phenyl]acetamide D45 (0.450 g; 2.5 mmol), cyclohexanone (0.245 g; 2.5 mmol) and H—Y zeolites (1 g) in methanol (20 ml) in a similar manner to that described in D3. ¹H NMR (CDCl₃) δ: 1.44-1.57 (4H, m), 1.71-1.73 (6H, m), 2.11 (1H, br s), 3.80 (3H, s), 4.64 (1H, s), 6.55 (1H, br s), 6.89-6.92 (2H, m), 7.36-7.40 (2H, m).

### Description 47

### 3-[4-(Methyloxy)phenyl]-1,4-diazaspiro[4.5]dec-3-en-2-one

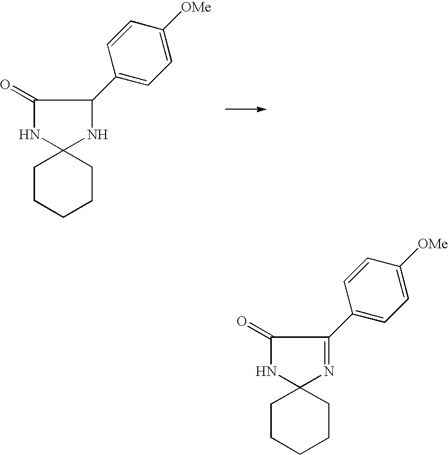

The title product (406 mg; 100%) was obtained from 3-[4-(methyloxy)phenyl]-1,4-diazaspiro[4.5]decan-2-one D46 (400 mg; 1.54 mmol) and N-bromosuccinimide (275 mg; 1.55 mmol) in DCM (20 ml) using a method similar to that described in D4. ¹H NMR (CDCl₃) δ: 1.40-1.75 (6H, m), 1.85-2.00 (4H, m), 3.87 (3H, s), 6.94-6.98 (2H, m), 8.18 (1H, br s), 8.37-8.40 (2H, m).

### Description 48

### 3-[3-Bromo-4-(methyloxy)phenyl]-1,4-diazaspiro[4.5]dec-3-en-2-one

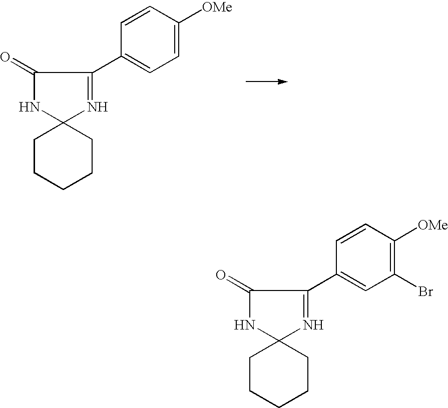

Bromine (0.25 ml; 4.86 mmol) was added to a stirred solution of 3-[4-(methyloxy)phenyl]-1,4-diazaspiro[4.5]dec-3-en-2-one D47 (1.21 g, 4.69 mmol) in DCM (30 ml) and the mixture stirred at room temperature for 1.5 h then heated at reflux for 5 h. The mixture was cooled and stirring continued for 88 h. Evaporation under reduced pressure, trituration with toluene (30 ml) and evaporation under reduced pressure gave a pale yellow solid (1.7 g). Purification of a 500 mg portion by MDAP gave the title compound (0.17 g). Mass Spectrum (Electrospray LC/MS): Found 337 (MH⁺). C₁₅H₁₇⁷⁹BrN₂O₂ requires 336. Ret. time 3.00 min.

Further bromo(substituted aryl)acetamides are either known in the literature or were prepared according to the method of description 33:

| Description | Structure | NMR | Name |
|---|---|---|---|
| 49 | 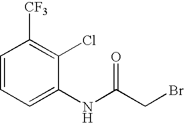 | ¹H NMR δ (CDCl₃,400 MHz) 4.10 (2H, s),7.40 (1H, m), 7.52 (1H,d), 8.58 (1H, d), 9.0(1H, s). | 2-bromo-N-[2-chloro-3-(trifluoromethyl)phenyl]acetamide |
| 50 | 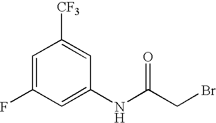 | ¹H NMR δ (CDCl₃,400 MHz) 4.09 2H, s),7.41 (1H, dd), 7.77(2H, m)., 11.0 (1H, s) | 2-bromo-N-[3-fluoro-5-(trifluoromethyl)phenyl]acetamide |
| 51 | 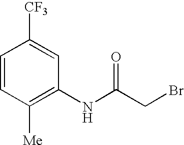 | ¹H NMR δ (CDCl₃,400 MHz), 2.31 (3H, s),4.14 (1H, s), 7.47 (2H,dd), 7.84 (1H, s), 9.9(1H, s). | 2-bromo-N-[2-methyl-5-(trifluoromethyl)phenyl]acetamide |
| 52 | 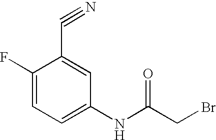 | ¹H NMR δ (CDCl₃,400 MHz), 4.05 (2H, s),7.21 (1H, m), 7.73 (1H,m), 7.95 (1H, q), 8.19(1H, s) | 2-bromo-N-(3-cyano-4-fluorophenyl)acetamide |
| 53 | 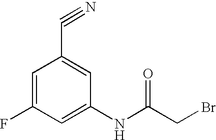 | ¹H NMR δ (CDCl₃,400 MHz) 4.06 (2H, s),7.63 (1H, s), 7.74 (1H,dd) 8.26 (1H, s) | 2-bromo-N-(3-cyano-5-fluorophenyl)acetamide |
| 54 | 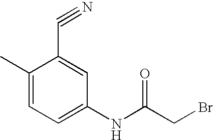 | ¹H NMR δ (CDCl₃,400 MHz) 2.53 (2H, s),7.31 (1H, d), 7.61 (1H,dd), 7.89 *1H d), 8.15(1H, s) | 2-bromo-N-(3-cyano-4-methylphenyl)acetamide |

### Example 1

### N-[2-(Methyloxy)phenyl]-2-{2-oxo-3-[4-(trifluoromethyl)phenyl]-1,4-diazaspiro[4.5]dec-3-en-1-yl}acetamide

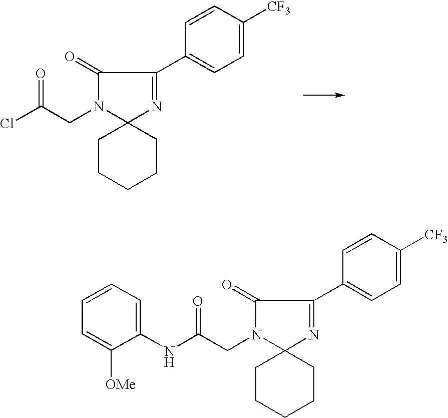

A solution of {2-oxo-3-[4-(trifluoromethyl)phenyl]-1,4-diazaspiro[4.5]dec-3-en-1-yl}acetyl chloride D7 (100 mg; 0.268 mmol) in DCM (2 ml) was added to a solution of 2-methoxyaniline (36 mg; 0.295 mmol) in DCM (2 ml) and triethylamine (0.075 ml; 0.537 mmol) and the mixture shaken for 66 h under argon. Saturated aqueous sodium hydrogen carbonate (8 ml) was added and shaking continued for 2 h. The organic layer was passed through a phase separation cartridge and the solvent removed under reduced pressure. The residue was purified by chromatography on silica gel eluting with 0-95% ethyl acetate in pentane gradient. Fractions containing desired product were pooled and solvent removed under reduced pressure to afford the title compound (88 mg; 72%). ¹H NMR (CDCl₃) δ: 1.20-1.55 (3H, m), 1.70-2.15 (7H, m), 3.81 (3H, s), 4.25 (2H, s), 6.85 (1H, d), 6.95 (1H, t), 7.05 (1H, t), 7.72 (2H, d), 8.25 (1H, d), 8.65 (3H, m). Mass Spectrum (Electrospray LC/MS) Found 460 (MH⁺). C₂₄H₂₄F₃N₃O₃ requires 459. Ret. Time 3.72 min.

### Example 2

### 2-[3-(4-Bromophenyl)-2-oxo-1,4-diazaspiro[4.5]dec-3-en-1-yl]-N-(3,4-difluorophenyl)acetamide

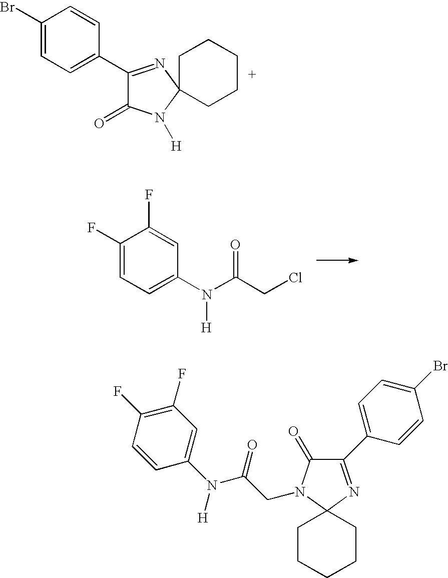

3-(4-Bromophenyl)-1,4-diazaspiro[4.5]dec-3-en-2-one D24 (0.500 g; 1.630 mmol) and 2-chloro-N-(3,4-difluorophenyl)acetamide D25 (0.375 g; 1.956 mmol) were dissolved in dry DMF (10 ml). Anhydrous potassium carbonate (0.529 g; 3.260 mmol) was added to the rapidly stirred solution, which was then heated to 60° C. for 16 h under an atmosphere of argon. After cooling, the reaction mixture was poured into water and extracted with ethyl acetate. The organic solution was washed with saturated brine, dried (MgSO₄) and evaporated at reduced pressure. The residue was chromatographed over silica gel (50 g), eluting with a gradient of 0 to 100% ethyl acetate-pentane. The title compound was obtained as a colourless oil (0.408 g; 53%). ¹H NMR (CDCl₃) δ: 1.30-1.45 (3H, m), 1.50-1.70 (1H, m), 1.78-2.15 (6H, m), 4.22 (2H, s), 7.08 (2H, m), 7.54-7.68 (3H, m), 8.38 (2H, d), 9.00 (1H, br s). Mass Spectrum (Electrospray LC/MS): Found 476 (MH⁺). C₂₂H₂₀⁷⁹BrF₂N₃O₂ requires 475. Ret. time 3.63 min.

### Example 3

### 2-[3-(4-Cyanophenyl)-2-oxo-1,4-diazaspiro[4.5]dec-3-en-1-yl]-N-(3,4-difluorophenyl)acetamide

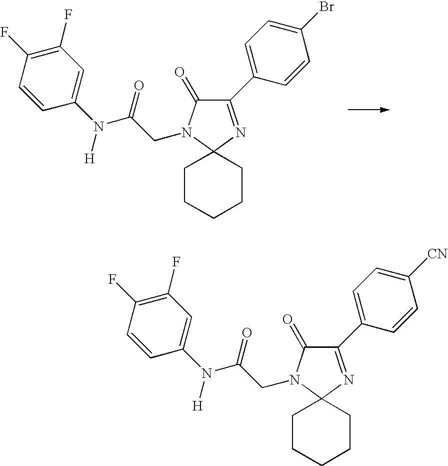

2-[3-(4-Bromophenyl)-2-oxo-1,4-diazaspiro[4.5]dec-3-en-1-yl]-N-(3,4-difluorophenyl)acetamide E2 (0.309 g; 0.649 mmol) was dissolved in NMP (5 ml) and heated to reflux with rapid stirring with copper(I) cyanide (0.117 g; 1.298 mmol) under an atmosphere of argon for 2 h. After cooling, ammonium hydroxide solution (10 ml; 4:1 water-0.88 ammonia solution) was added and the aqueous solution extracted with ethyl acetate. The organic solution was washed with water, saturated brine, dried (MgSO₄) and evaporated at reduced pressure. The residue was chromatographed over silica gel (20 g). Elution with a gradient of 0 to 50% ethyl acetate-pentane gave the title compound as a pale yellow foam (0.204 g; 74%). ¹H NMR (CDCl₃) δ: 1.32-1.48 (3H, m), 1.80-2.16 (7H, m), 4.22 (2H, s), 7.08 (2H, m), 7.55-7.62 (1H, m), 7.79 (2H, d), 8.60 (2H, d), 8.90 (1H, br s). Mass Spectrum (Electrospray LC/MS): Found 423 (MH⁺). C₂₃H₂₀F₂N₄O₂ requires 422. Ret. time 3.36 min.

The compounds in the table below were prepared using similar methods to those described for the Examples above. Method: A=Acid chloride (using method similar to that in Example 1); B=Alkylation (using method similar to that in Example 2). Work-up and purification was carried out using appropriate methods similar to those described in the examples above.

Anilines and arylglycine starting materials were obtained commercially.

| | | | Mass spectrum | |
|---|---|---|---|---|
| | | | (Electrospray | |
| | | | LC/MS), APl⁺ | |
| Ex | Structure | Method | Ret. time (min) | Name |
| 4 | 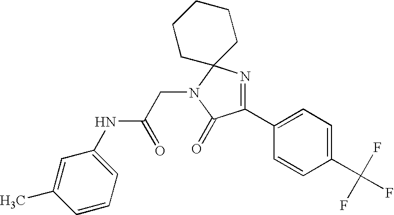 | A | Found 444 (MH⁺)C₂₄H₂₄F₃N₃O₂requires 443;3.79. | N-(3-methylphenyl)-2-{2-oxo-3-[4-(trifluoromethyl)phenyl]-1,4-diazaspiro[4.5]dec-3-en-1-yl}acetamide |
| 5 | 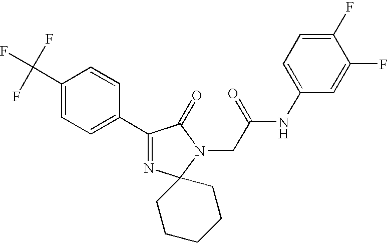 | A | Found 466 (MH⁺)C₂₃H₂₀F₅N₃O₂requires 465;3.71. | N-(3,4-difluorophenyl)-2-{2-oxo-3-[4-(trifluoromethyl)phenyl]-1,4-diazaspiro[4.5]dec-3-en-1-yl}acetamide |
| 6 | 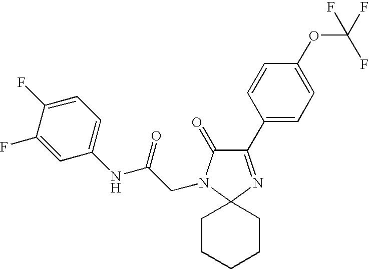 | A | Found 482 (MH⁺)C₂₃H₂₀F₅N₃O₃requires 481;3.78. | N-(3,4-difluorophenyl)-2-(2-oxo-3-{4-[(trifluoromethyl)oxy]phenyl}-1,4-diazaspiro[4.5]dec-3-en-1-yl)acetamide |
| 7 | 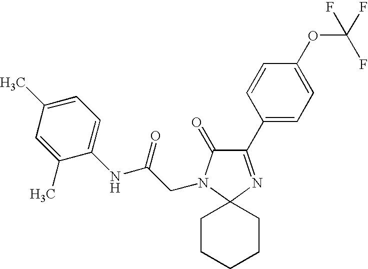 | A | Found 474 (MH⁺)C₂₅H₂₆F₃N₃O₃requires 473;3.81. | N-(2,4-dimethylphenyl)-2-(2-oxo-3-{4-[(trifluoromethyl)oxy]phenyl}-1,4-diazaspiro[4.5]dec-3-en-1-yl)acetamide |
| 8 | 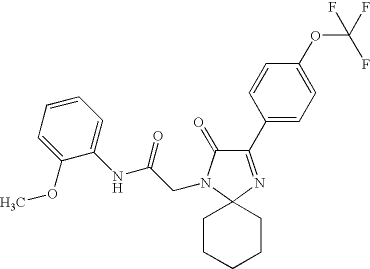 | A | Found 476 (MH⁺)C₂₄H₂₄F₃N₃O₄requires 475;3.80. | N-[2-(methyloxy)phenyl]-2-(2-oxo-3-{4-[(trifluoromethyl)oxy]phenyl}-1,4-diazaspiro[4.5]dec-3-en-1-yl)acetamide |
| 9 | 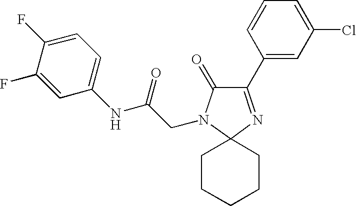 | A | Found 432 (MH⁺)C₂₂H₂₀³⁵ClF₂N₃O₂requires 431;3.70. | 2-[3-(3-chlorophenyl)-2-oxo-1,4-diazaspiro[4.5]dec-3-en-1-yl]-N-(3,4-difluorophenyl)acetamide |
| 10 | 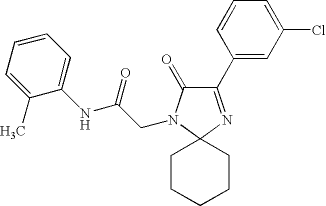 | A | Found 410 (MH⁺)C₂₃H₂₄³⁵ClN₃O₂requires 409;3.61. | 2-[3-(3-chlorophenyl)-2-oxo-1,4-diazaspiro[4.5]dec-3-en-1-yl]-N-(2-methylphenyl)acetamide |
| 11 | 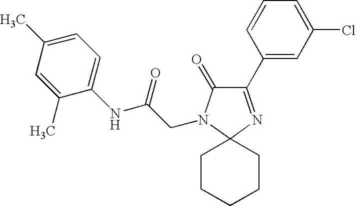 | A | Found 424 (MH⁺)C₂₄H₂₆³⁵ClN₃O₂requires 423;3.73. | 2-[3-(3-chlorophenyl)-2-oxo-1,4-diazaspiro[4.5]dec-3-en-1-yl]-N-(2,4-dimethylphenyl)acetamide |
| 12 | 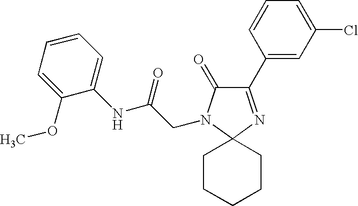 | A | Found 426 (MH⁺)C₂₃H₂₄³⁵ClN₃O₃requires 425;3.71. | 2-[3-(3-chlorophenyl)-2-oxo-1,4-diazaspiro[4.5]dec-3-en-1-yl]-N-[2-(methyloxy)phenyl]acetamide |
| 13 | 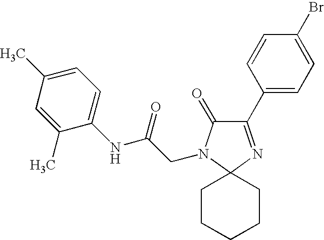 | B | Found 468 (MH⁺)C₂₄H₂₆⁷⁹BrN₃O₂requires 467;3.76. | 2-[3-(4-bromophenyl)-2-oxo-1,4-diazaspiro[4.5]dec-3-en-1-yl]-N-(2,4-dimethylphenyl)acetamide |
| 14 | 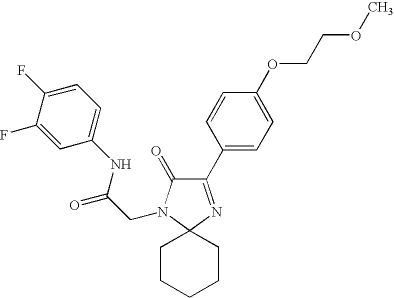 | B | Found 472 (MH⁺)C₂₅H₂₇F₂N₃O₄requires 471;3.33. | N-(3,4-difluorophenyl)-2-[3-(4-{[2-(methyloxy)ethyl]oxy}phenyl)-2-oxo-1,4-diazaspiro[4.5]dec-3-en-1-yl]acetamide |

### Example 15

### 2-[3-(4-Cyanophenyl)-2-oxo-1,4-diazaspiro[4.4]non-3-en-1-yl]-N-(3,5-difluorophenyl)acetamide

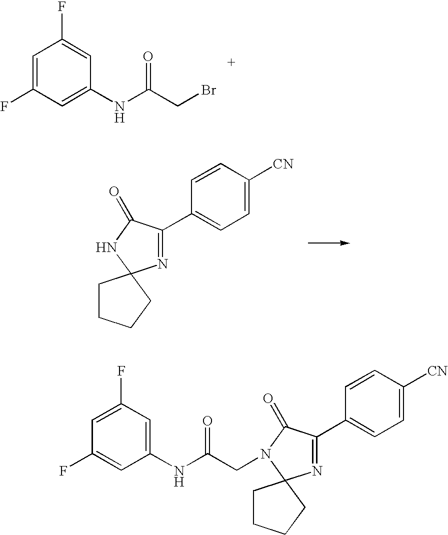

60% Sodium hydride in oil (20 mg; 0.5 mmol) was added in one portion to a stirred solution of 4-(3-oxo-1,4-diazaspiro[4.4]non-1-en-2-yl)benzonitrile D36 (100 mg; 0.42 mmol) in DMF (3 ml) under argon. After stirring for 5 minutes 2-bromo-N-(3,5-difluorophenyl)acetamide D33 (86 mg; 0.36 mmol) was added in one portion. After a further 3 hours, saturated aqueous sodium hydrogen carbonate (50 ml) was added. After 18 hours further saturated aqueous sodium hydrogen carbonate (200 ml) was added and the mixture extracted with ethyl acetate (2×150 ml). Combined organics were dried (Na₂SO₄) and evaporated under reduced pressure. The residue was dissolved in DMSO to give 1.8 ml of solution and purified in 2 portions using mass directed auto-purification chromatography to afford the title product (95 mg; 65%). ¹H NMR (CDCl₃) δ: 1.80-1.95 (2H, m), 2.00-2.25 (6H, m), 4.25 (2H, s), 6.52-6.59 (1H, m), 7.08-7.15 (2H, m), 7.76-7.80 (2H, m), 8.55-8.59 (2H, m), 9.05 (1H, s). Mass Spectrum (Electrospray LC/MS): Found 409 (MH⁺). C₂₂H₁₈F₂N₄O₂ requires 408. Ret. time 3.22 min.

### Alternative Preparation

60% Sodium hydride in oil (0.2 g; 5 mmol) was added in one portion to a stirred solution of 4-(3-oxo-1,4-diazaspiro[4.4]non-1-en-2-yl)benzonitrile D36 (1 g; 4.18 mmol) in anhydrous DMF (20 ml) under argon. After stirring for at room temperature for 10 minutes, 2-bromo-N-(3,5-difluorophenyl)acetamide D33 (1.25 g; 5 mmol) was added in one portion and stirred at room temperature. After a further 2 hours, saturated aqueous sodium hydrogen carbonate (400 ml) was added and the reaction mixture was allowed to stand at room temperature overnight. The mixture was extracted with ethyl acetate (2×300 ml). Combined organics were washed with brine (300 ml), dried (Na₂SO₄) and evaporated under reduced pressure to afford a brown solid. Purification by SP4 chromatography using 0-70% ethyl acetate/pentane gave mixed fractions, therefore fractions containing desired product were pooled and evaporated under reduced pressure. Chromatography on SP4 (40M silica column) eluting with 0-40% ethyl acetate/pentane gave the title product (700 mg) ¹H NMR (CDCl₃) δ: 1.80-1.95 (2H, m), 2.00-2.25 (6H, m), 4.25 (2H, s), 6.52-6.59 (1H, m), 7.08-7.15 (2H, m), 7.76-7.80 (2H, m), 8.55-8.59 (2H, m), 9.05 (1H, s). Mass Spectrum (Electrospray LC/MS): Found 409 (MH⁺). C₂₂H₁₈F₂N₄O₂ requires 408. Ret. time 3.19 min.

### Example 16

### N-(2-Chlorophenyl)-2-{2-oxo-3-[4-(trifluoromethyl)phenyl]-1,4-diazaspiro[4.4]non-3-en-1-yl}acetamide

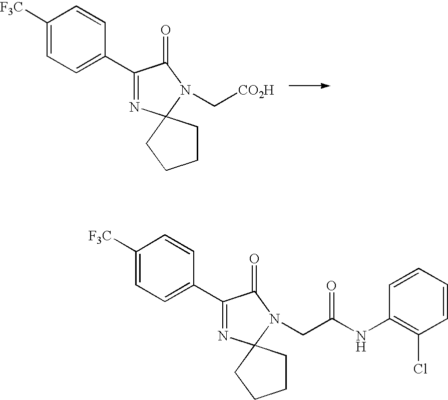

Diisopropylethylamine (16.5 uL, 0.095 mmol) and 2-chloroaniline (11 uL, 0.104 mmol) were added to a stirred solution of {2-oxo-3-[4-(trifluoromethyl)phenyl]-1,4-diazaspiro[4.4]non-3-en-1-yl}acetic acid D44 (32 mg, 0.095 mmol) in DCM (1 ml). HATU was then added as a solution in DMF (800 mg/ml, 50 ul, 0.104 mmol). The mixture was then left to stir at room temperature for 46 h. More DCM was added and then more diisopropylethylamine (16.5 ul, 0.095 mmol), 2-chloroaniline (11 ul, 0.104 mmol) and HATU (as a solid, 40 mg, 0.104) were added and the mixture left to stir for a further 2.5 h. The mixture was concentrated by flow of N₂ gas and then purified by mass directed auto-purification chromatography (Method 2) to give the title compound (26 mg, 61%); ¹H NMR (d₆-DMSO) δ: 1.67-1.76 (2H, m), 1.89-2.06 (4H, m), 2.12-2.21 (2H, m), 4.43 (2H, s), 7.22 (1H, t), 7.34 (1H, t), 7.52 (1H, d), 7.71 (1H, d), 7.92 (2H, d), 8.55 (2H, d), 9.76 (1H, br s).

### Example 17

### N-(3,4-Difluorophenyl)-2-{2-oxo-3-[4-(trifluoromethyl)phenyl]-1,4-diazaspiro[4.4]non-3-en-1-yl}acetamide

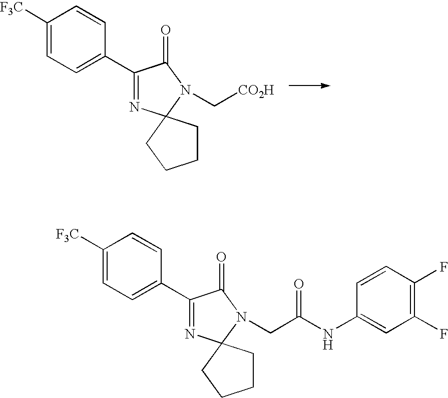

Diisopropylethylamine (12.7 ul, 0.073 mmol) and 3,4-difluoroaniline (7.9 ul, 0.080 mmol) were added to a stirred solution of {2-oxo-3-[4-(trifluoromethyl)phenyl]-1,4-diazaspiro[4.4]non-3-en-1-yl}acetic acid D44 (25 mg, 0.073 mmol) in DCM (1 ml). HATU (31 mg, 0.080) was then added and the mixture was left to stir at room temperature for 62.5 h. The mixture was concentrated by flow of N₂ gas and then purified twice, first by UV-directed preparative HPLC purification chromatography and then by mass directed auto-purification chromatography (Method 2) to give the title compound (19.5 mg, 59%); ¹H NMR (CDCl₃) δ: 1.82-1.94 (2H, m), 2.01-2.24 (6H, m), 4.25 (2H, s), 7.05-7.11 (2H, m), 7.56-7.64 (1H, m), 7.76 (2H, d), 8.57 (2H, d), 8.94 (1H, br s).

### Example 18

### N-(3,5-Difluorophenyl)-2-{2-oxo-3-[4-(trifluoromethyl)phenyl]-1,4-diazaspiro[4.4]non-3-en-1-yl}acetamide

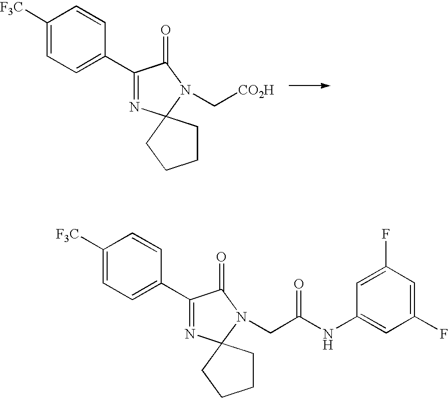

Diisopropylethylamine (12.7 ul, 0.073 mmol) and 3,5-difluoroaniline (11 mg, 0.080 mmol) were added to a stirred solution of {2-oxo-3-[4-(trifluoromethyl)phenyl]-1,4-diazaspiro[4.4]non-3-en-1-yl}acetic acid D44 (25 mg, 0.073 mmol) in DCM (1 ml). HATU (31 mg, 0.080) was then added and the mixture was left to stir at room temperature for 62.5 h. The mixture was concentrated by flow of N₂ gas and then purified twice, first by UV-directed preparative HPLC purification chromatography and then by mass directed auto-purification chromatography (Method 2) to give the title compound (19.5 mg, 59%); ¹H NMR (CDCl3) δ: 1.83-1.94 (2H, m), 2.00-2.24 (6H, m), 4.25 (2H, s), 6.56 (1H, tt), 7.12 (2H, dd), 7.76 (2H, d), 8.57 (2H, d), 9.10 (1H, br s).

The compounds in the table below were prepared using similar methods to those described for the Examples above. Method: A=Acid chloride (using method similar to that in Example 1); B=Alkylation (using method similar to that in Example 2); C=Cyanation (using method similar to that in Example 3). Work-up and purification was carried out using appropriate methods similar to those described in the examples above.

Anilines, and arylglycine starting materials were either obtained commercially or prepared by literature methods.

| | | | Mass spectrum | |
|---|---|---|---|---|
| | | | (Electrospray | |
| | | | LC/MS), API⁺ | |
| Ex | Structure | Method | Ret. time (min) | Name |
| 19 | 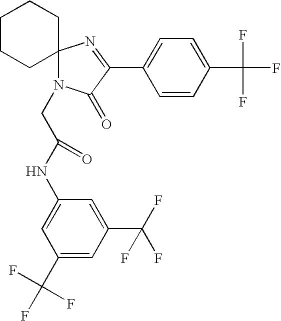 | A | Found 566 (MH⁺)C₂₅H₂₀F₉N₃O₂requires 565;4.05. | N-[3,5-bis(trifluoromethyl)phenyl]-2-{2-oxo-3-[4-(trifluoromethyl)phenyl]-1,4-diazaspiro[4.5]dec-3-en-1-yl}acetamide |
| 20 | 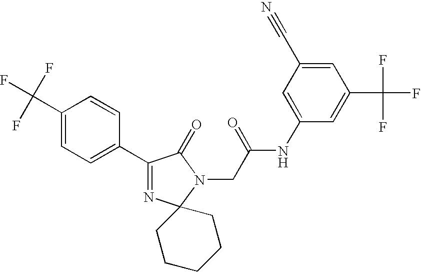 | A | Found 523 (MH⁺)C₂₅H₂₀F₆N₄O₂requires 522;3.82. | N-[3-cyano-5-(trifluoromethyl)phenyl]-2-{2-oxo-3-[4-(trifluoromethyl)phenyl]-1,4-diazaspiro[4.5]dec-3-en-1-yl}acetamide |
| 21 | 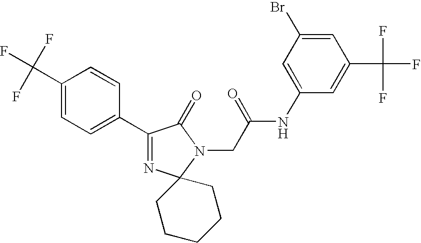 | A | Found 576 (MH⁺)C₂₄H₂₀⁷⁹BrF₆N₃O₂requires 575;4.07. | N-[3-bromo-5-(trifluoromethyl)phenyl]-2-{2-oxo-3-[4-(trifluoromethyl)phenyl]-1,4-diazaspiro[4.5]dec-3-en-1-yl}acetamide |
| 22 | 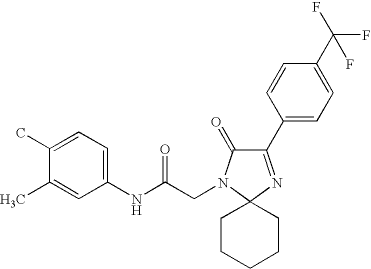 | A | Found 478 (MH⁺)C₂₄H₂₃³⁵ClF₃N₃O₂requires 477;3.85. | N-(4-chloro-3-methylphenyl)-2-{2-oxo-3-[4-(trifluoromethyl)phenyl]-1,4-diazaspiro[4.5]dec-3-en-1-yl}acetamide |
| 23 | 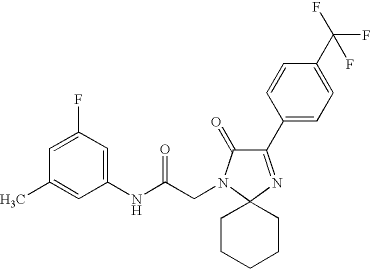 | A | Found 462 (MH⁺)C₂₄H₂₃F₄N₃O₂requires 461;3.74. | N-(3-fluoro-5-methylphenyl)-2-{2-oxo-3-[4-(trifluoromethyl)phenyl]-1,4-diazaspiro[4.5]dec-3-en-1-yl}acetamide |
| 24 | 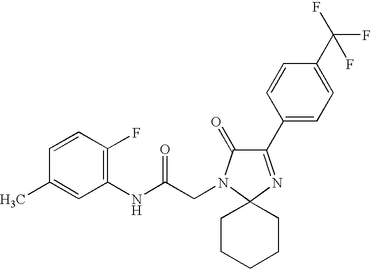 | A | Found 462 (MH⁺)C₂₄H₂₃F₄N₃O₂requires 461;3.73. | N-(2-fluoro-5-methylphenyl)-2-{2-oxo-3-[4-(trifluoromethyl)phenyl]-1,4-diazaspiro[4.5]dec-3-en-1-yl}acetamide |
| 25 | 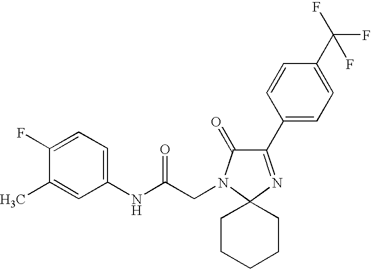 | A | Found 462 (MH⁺)C₂₄H₂₃F₄N₃O₂requires 461;3.68. | N-(4-fluoro-3-methylphenyl)-2-{2-oxo-3-[4-(trifluoromethyl)phenyl]-1,4-diazaspiro[4.5]dec-3-en-1-yl}acetamide |
| 26 | 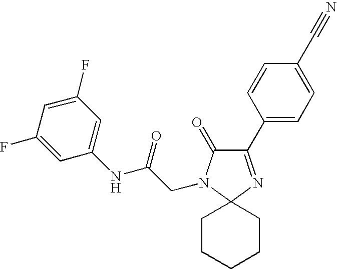 | A | Found 423 (MH⁺)C₂₃H₂₀F₂N₄O₂requires 422;3.44. | 2-[3-(4-cyanophenyl)-2-oxo-1,4-diazaspiro[4.5]dec-3-en-1-yl]-N-(3,5-difluorophenyl)acetamide |
| 27 | 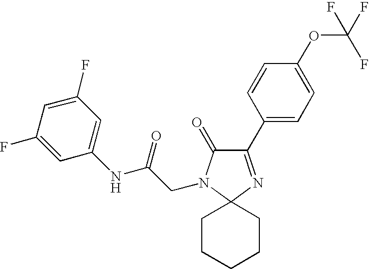 | A | Found 482 (MH⁺)C₂₃H₂₀F₅N₃O₃requires 481;3.82. | N-(3,5-difluorophenyl)-2-(2-oxo-3-{4-[(trifluoromethyl)oxy]phenyl}-1,4-diazaspiro[4.5]dec-3-en-1-yl)acetamide |
| 28 | 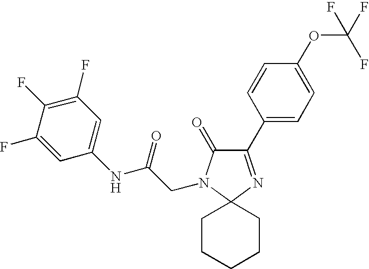 | A | Found 500 (MH⁺)C₂₃H₁₉F₆N₃O₃requires 499;3.87. | 2-(2-oxo-3-{4-[(trifluoromethyl)oxy]phenyl}-1,4-diazaspiro[4.5]dec-3-en-1-yl)-N-(3,4,5-trifluorophenyl)acetamide |
| 29 | 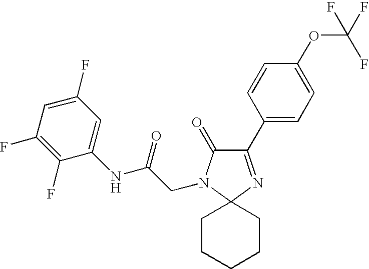 | A | Found 500 (MH⁺)C₂₃H₁₉F₆N₃O₃requires 499;3.88. | 2-(2-oxo-3-{4-[(trifluoromethyl)oxy]phenyl}-1,4-diazaspiro[4.5]dec-3-en-1-yl)-N-(2,3,5-trifluorophenyl)acetamide |
| 30 | 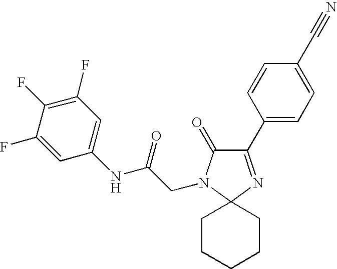 | A | Found 441 (MH⁺)C₂₃H₁₉F₃N₄O₂requires 440;3.46. | 2-[3-(4-cyanophenyl)-2-oxo-1,4-diazaspiro[4.5]dec-3-en-1-yl]-N-(3,4,5-trifluorophenyl)acetamide |
| 31 | 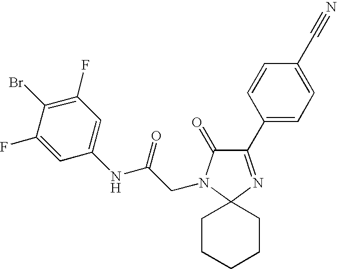 | A | Found 501 (MH⁺)C₂₃H₁₉⁷⁹BrF₂N₄O₂requires 500;3.60. | N-(4-bromo-3,5-difluorophenyl)-2-[3-(4-cyanophenyl)-2-oxo-1,4-diazaspiro[4.5]dec-3-en-1-yl]acetamide |
| 32 | 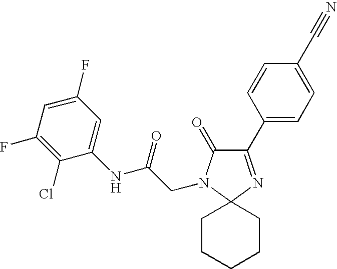 | A | Found 457 (MH⁺)C₂₃H₁₉³⁵ClF₂N₄O₂requires 456;3.59. | N-(2-chloro-3,5-difluorophenyl)-2-[3-(4-cyanophenyl)-2-oxo-1,4-diazaspiro[4.5]dec-3-en-1-yl]acetamide |
| 33 | 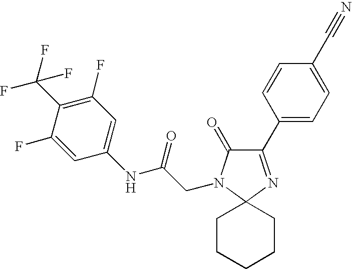 | A | Found 491 (MH⁺)C₂₄H₁₉F₅N₄O₂requires 490;3.65. | 2-[3-(4-cyanophenyl)-2-oxo-1,4-diazaspiro[4.5]dec-3-en-1-yl]-N-[3,5-difluoro-4-(trifluoromethyl)phenyl]acetamide |
| 34 | 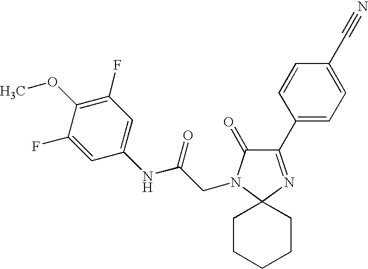 | A | Found 453 (MH⁺)C₂₄H₂₂F₂N₄O₃requires 452;3.38. | 2-[3-(4-cyanophenyl)-2-oxo-1,4-diazaspiro[4.5]dec-3-en-1-yl]-N-[3,5-difluoro-4-(methyloxy)phenyl]acetamide |
| 35 | 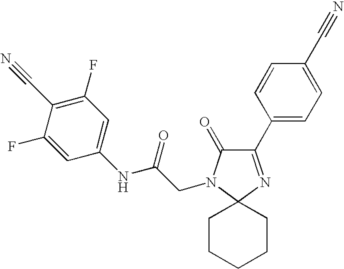 | C | Found 448 (MH⁺)C₂₄H₁₉F₂N₅O_{O}₂requires 447;3.38. | N-(4-cyano-3,5-difluorophenyl)-2-[3-(4-cyanophenyl)-2-oxo-1,4-diazaspiro[4.5]dec-3-en-1-yl]acetamide |
| 36 | 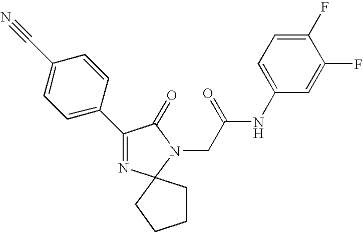 | B | Found 409 (MH⁺)C₂₂H₁₈F₂N₄O₂requires 408;3.18. | 2-[3-(4-cyanophenyl)-2-oxo-1,4-diazaspiro[4.4]non-3-en-1-yl]-N-(3,4-difluorophenyl)acetamide |
| 37 | 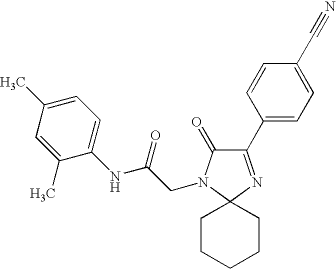 | C | Found 415 (MH⁺)C₂₅H₂₆N₄O₂requires 414;3.34. | 2-[3-(4-cyanophenyl)-2-oxo-1,4-diazaspiro [4,5]dec-3-en-1-yl]-N-(2,4-dimethylphenyl)acetamide |
| 38 | 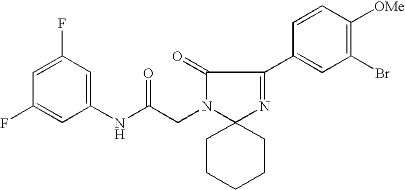 | B | Found 506 (MH⁺)C₂₃H₂₂⁷⁹BrF_{2N}₃O₃requires 505;3.71. | 2-{3-[3-bromo-4-(methyloxy)phenyl]-2-oxo-1,4-diazaspiro[4.5]dec-3-en-1-yl}-N-(3,5-difluorophenyl)acetamide |
| 39 | 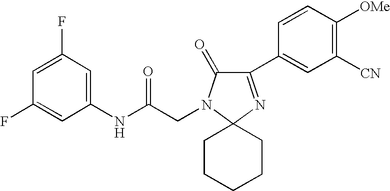 | C | Found 453 (MH⁺)C₂₄H₂₂F₂N₄O₃requires 452;3.48. | 2-{3-[3-cyano-4-(methyloxy)phenyl]-2-oxo-1,4-diazaspiro[4.5]dec-3-en-1-yl}-N-(3,5-difluorophenyl)acetamide |

3-Amino-5-(trifluoromethyl)benzonitrile was prepared as described by G. Butora et al. PCT WO2004/041161A2.

### Example 40

### 2-[3-(4-cyanophenyl)-2-oxo-1,4-diazaspiro[4.4]non-3-en-1-yl]-N-[3-(trifluoromethyl)phenyl]acetamide

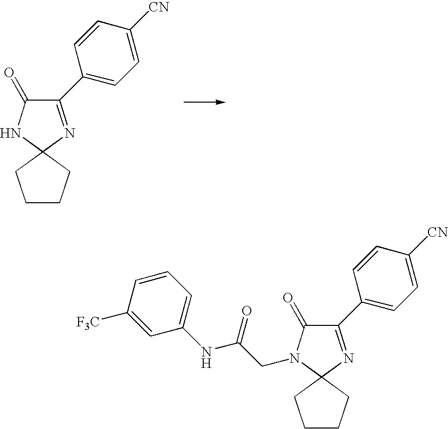

To a solution of 4-(3-oxo-1,4-diazaspiro[4.4]non-1-en-2-yl)benzonitrile D36 (1.0 eq., 100 mg, 0.418 mmol,) in dimethylformamide (2 ml), 60% sodium hydride dispersion in mineral oil (1.1 eq., 18 mg) was added at 0° C. The reaction was stirred for 20 minutes at r.t. 2-Bromo-N-[3-(trifluoromethyl)phenyl]acetamide (1.2 eq., 141 mg, 0.502 mmol) dissolved in dimethylformamide (2 ml) was added by syringe pump over 30 minutes. The reaction was left stirring for 4 hours at r.t. Water (50 ml) was added and the aqueous layer extracted with ethyl acetate (3×50 ml); organics were alternatively washed with water (2×30 ml) and brine (2×30 ml). The organics were combined and dried over Na₂SO₄, filtered and the solvent was evaporated to afford the crude product. The crude product was purified by column chromatography (Ethyl acetate in hexane eluant) on silica column to yield the title compound, (56 mg, 25%). Found 441 (MH⁺). C₂₃H₁₉F₃N₄O₂ requires 440. Ret. Time 3.20 min. ¹H NMR δ (DMSO, 400 MHz) 1.70 (2H, m), 1.94 (4H, m), 2.16 (2H, m), 4.38 (2H, s), 7.44 (1H, d), 7.58 (1H, t), 7.75 (1H, d), 8.02 (2H, d), 8.10 (1H, s), 8.50 (2H, d), 10.48 (1H, br. S).

### Alternative Preparation

To a solution of 4-(3-oxo-1,4-diazaspiro[4.4]non-1-en-2-yl)benzonitrile D36 (1.0 eq., 700 mg, 2.93 mmol) in dimethylformamide (14 ml), 60% sodium hydride dispersion in mineral oil (1.1 eq., 128 mg, 5.37 mmol) was added at 0° C. The reaction was stirred for 20 minutes at r.t. 2-Bromo-N-[3-(trifluoromethyl)phenyl]acetamide (1.2 eq., 987 mg, 3.52 mmol) dissolved in dimethylformamide (14 ml) was added by syringe pump over 30 minutes. The reaction was left stirring over the weekend at r.t. Water (50 ml) was added and the aqueous layer extracted with ethyl acetate (2×50 ml); organics were combined alternatively, washed with water (2×50 ml) and brine (2×50 ml). The organics were dried over Na₂SO₄, filtered and the solvent was evaporated to afford the crude product. The crude product was purified by column chromatography (Ethyl acetate in hexane eluant) on silica column followed by MDAP to yield the title compound, (437 mg, 34%). Found 441 (MH⁺). C₂₃H₁₉F₃N₄O₂ requires 440. Ret. Time 3.16 min. ¹H NMR δ (DMSO, 400 MHz) 1.71 (2H, m), 1.94 (4H, m), 2.16 (2H, m), 4.38 (2H, s), 7.44 (1H, d), 7.58 (1H, t), 7.75 (1H, d), 8.01 (2H, d), 8.10 (1H, s), 8.50 (2H, d), 10.47 (1H, br. S).

### Example 41

### 2-[3-(4-cyanophenyl)-2-oxo-1,4-diazaspiro[4.5]dec-3-en-1-yl]-N-[3-(trifluoromethyl)phenyl]acetamide

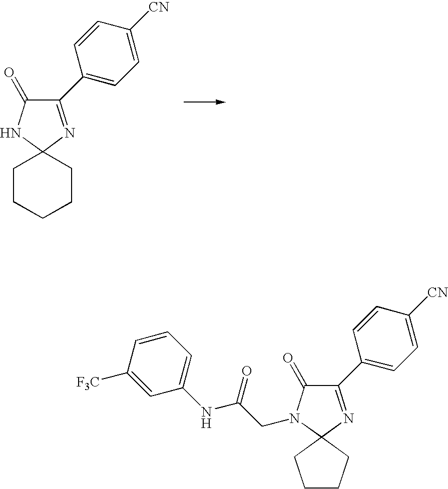

To a solution of 4-(3-oxo-1,4-diazaspiro[4.5]dec-1-en-2-yl)benzonitrile D37 (1.0 eq., 0.395 mmol, 100 mg) in dimethylformamide (2 ml), 60% sodium hydride dispersion in mineral oil (1.1 eq., 17 mg) added at 0° C. The reaction was stirred for 20 minutes at r.t. 2-bromo-N-[3-(trifluoromethyl)phenyl]acetamide (1.2 eq., 0.474 mmol, 133 mg) dissolved in dimethylformamide (2 ml) was added by syringe pump over 30 minutes. The reaction was left stirring for 4 hours at r.t. Water (30 ml) added and the aqueous layer extracted with ethyl acetate (2×30 ml); organics were alternatively washed with water (2×30 ml) and brine (2×30 ml). The organics were combined dried over Na₂SO₄, filtered and the solvent was evaporated to afford the crude product. The crude product was purified by column chromatography (Ethyl acetate in hexane) on silica column to yield the title compound (71 mg, 40%)

Mass Spectrum (Electrospray LC/MS): Found 455 (MH⁺). C₂₄H₂₁F₃N₄O₂ requires 454. Ret. Time 3.39 min.

¹H NMR δ (DMSO, 400 MHz) 1.38 (3H, m), 1.76 (3H, m), 1.891 (2H, q), 2.034 (2H, m), 4.376 (2H, s), 7.434 (1H, d), 7.579 (1H, t), 7.747 (1H, d), 8.031 (2H, m), 8.095 (1H, s), 8.521 (2H, m), 10.55 (1H, br. S).

The compounds in the table below were prepared using similar methods to those described for the Examples above. Work-up was carried out using appropriate methods similar to those described in the examples above. Purification methods were as follows A=Column chromatography (Ethyl acetate in hexane) on silica column; B=Mass Directed Auto Preparation. A gradient using acetonitrile/water and formic acid as a modifier; C=Column chromatography (Ethyl acetate in hexane) on silica column followed by Mass Directed Auto Preparation, a gradient using acetonitrile/water and formic acid as a modifier.

| | | | Mass spectrum | |
|---|---|---|---|---|
| | | | (Electrospray | Purifi- |
| Example | Compound | | LC/MS), API⁺ | cation |
| No | Name | Structure | Ret. time (min) | method |
| 42 | 2-[3-(4-cyanophenyl)-2-oxo-1,4-diazaspiro[4.4]non-3-en-1-yl]-N-[4-fluoro-3-(trifluoromethyl)phenyl]acetamide | 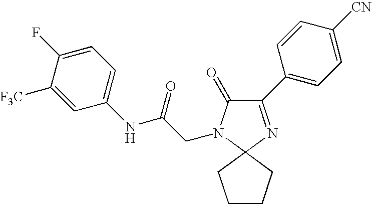 | Found 459 (MH⁺).C₂₃H₁₈F₄N₄O₂Requires 458.4;3.24 | A |
| 43 | N-[2-chloro-3-(trifluoromethyl)phenyl]-2-[3-(4-cyanophenyl)-2-oxo-1,4-diazaspiro[4.4]non-3-en-1-yl]acetamide | 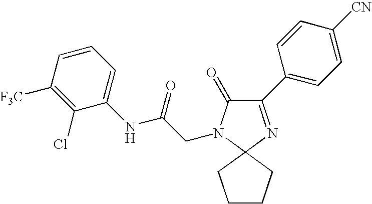 | Found 475 (MH⁺).C₂₃H₁₈³⁵ClF₃N₄O₂Requires 474.9;3.32 | A |
| 44 | N-[4-chloro-3-(trifluoromethyl)phenyl]-2-[3-(4-cyanophenyl)-2-oxo-1,4-diazaspiro[4.4]non-3-en-1-yl]acetamide | 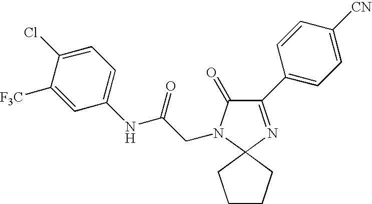 | Found 475 (MH⁺).C₂₃H₁₈³⁵ClF₃N₄O₂Requires 474.9;3.36 | A |
| 45 | 2-[3-(4-cyanophenyl)-2-oxo-1,4-diazaspiro[4.5]dec-3-en-1-yl]-N-[4-fluoro-3-(trifluoromethyl)phenyl]acetamide | 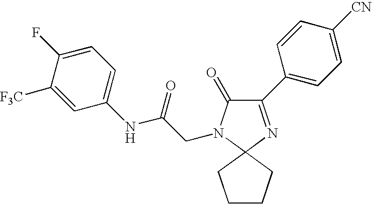 | Found 473 (MH⁺).C₂₄H₂₀F₄N₄O₂Requires 472.4;3.41 | A |
| 46 | N-[2-chloro-3-(trifluoromethyl)phenyl]-2-[3-(4-cyanophenyl)-2-oxo-1,4-diazaspiro[4.5]dec-3-en-1-yl]acetamide | 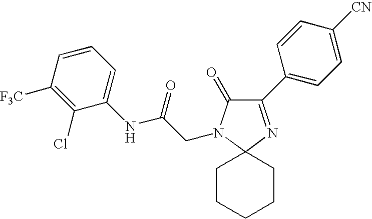 | Found 489 (MH⁺).C₂₄H₂₀³⁵ClF₃N₄O₂Requires 488;3.50 | C |
| 47 | 2-[3-(4-cyanophenyl)-2-oxo-1,4-diazaspiro[4.4]non-3-en-1-yl]-N-[3-fluoro-5-(trifluoromethyl)phenyl]acetamide | 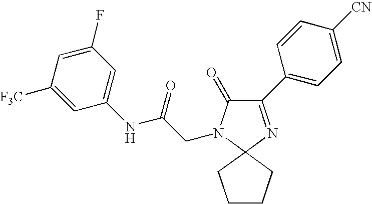 | Found 459 (MH⁺).C₂₃H₁₈F₄N₄O₂Requires 458.4;3.29 | C |
| 48 | 2-[3-(4-cyanophenyl)-2-oxo-1,4-diazaspiro[4.5]dec-3-en-1-yl]-N-[3-fluoro-5-(trifluoromethyl)phenyl]acetamide | 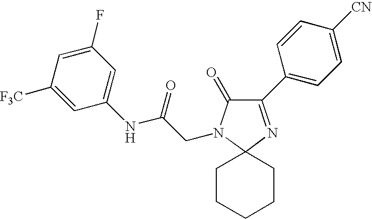 | Found 473 (MH⁺).C₂₄H₂₀F₄N₄O₂Requires 472.4;3.47 | B |
| 49 | 2-[3-(4-cyanophenyl)-2-oxo-1,4-diazaspiro[4.4]non-3-en-1-yl]-N-[2-methyl-5-(trifluoromethyl)phenyl]acetamide | 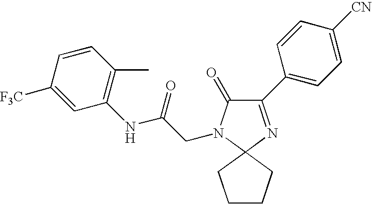 | Found 455 (MH⁺).C₂₄H₂₁F₃N₄O₂Requires 454.4;1.29 | C |
| 50 | 2-[3-(4-cyanophenyl)-2-oxo-1,4-diazaspiro[4.5]dec-3-en-1-yl]-N-[2-methyl-5-(trifluoromethyl)phenyl]acetamide | 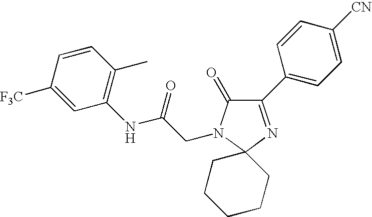 | Found 469 (MH⁺).C₂₅H₂₃F₃N₄O₂Requires 468.5;1.37 | B |
| 51 | N-[4-chloro-3-(trifluoromethyl)phenyl]-2-[3-(4-cyanophenyl)-2-oxo-1,4-diazaspiro[4.5]dec-3-en-1-yl]acetamide | 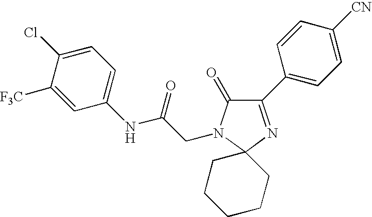 | Found 489 (MH⁺).C₂₄H₂₀³⁵ClF₃N₄O₂Requires 488.;3.61 | B |
| 52 | N-[2-chloro-5-(trifluoromethyl)phenyl]-2-[3-(4-cyanophenyl)-2-oxo-1,4-diazaspiro[4.4]non-3-en-1-yl]acetamide | 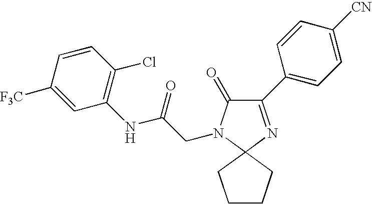 | Found 475 (MH⁺).C₂₃H₁₈³⁵ClF₃N₄O₂Requires 474.9;3.45 | B |
| 53 | N-[2-chloro-5-(trifluoromethyl)phenyl]-2-[3-(4-cyanophenyl)-2-oxo-1,4-diazaspiro[4.5]dec-3-en-1-yl]acetamide | 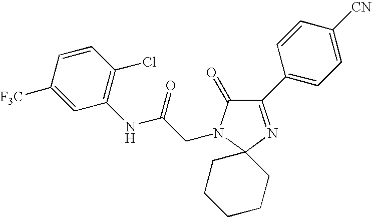 | Found 489 (MH⁺).C₂₄H₂₀³⁵ClF₃N₄O₂Requires 488.0;1.44 | B |
| 54 | N-(3-cyanophenyl)-2-[3-(4-cyanophenyl)-2-oxo-1,4-diazaspiro[4.4]non-3-en-1-yl]acetamide | 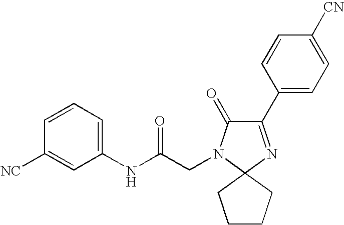 | Found 398 (MH⁺).C₂₃H₁₉N₅O₂Requires 397.4;2.89 | B |
| 55 | N-(3-cyanophenyl)-2-[3-(4-cyanophenyl)-2-oxo-1,4-diazaspiro[4.5]dec-3-en-1-yl]acetamide | 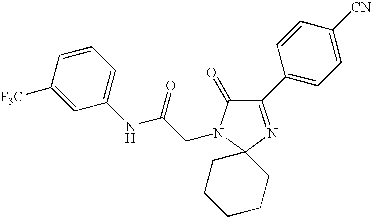 | Found 412 (MH⁺).C₂₄H₂₁N₅O₂Requires 411.4;3.07 | B |
| 56 | N-(3-cyano-4-fluorophenyl)-2-[3-(4-cyanophenyl)-2-oxo-1,4-diazaspiro[4.5]dec-3-en-1-yl]acetamide | 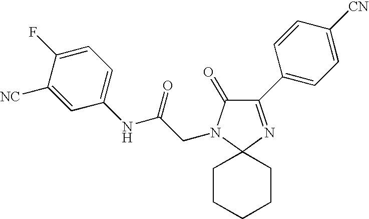 | Found 430 (MH⁺).C₂₄H₂₀FN₅O₂Requires 429.4;3.13 | B |
| 57 | N-(3-cyano-5-fluorophenyl)-2-[3-(4-cyanophenyl)-2-oxo-1,4-diazaspiro[4.5]dec-3-en-1-yl]acetamide | 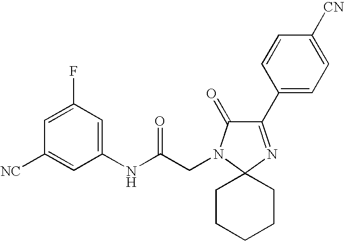 | Found 430 (MH⁺).C₂₄H₂₀FN₅O₂Requires 429.5;3.26 | B |
| 58 | N-(3-cyano-4-methylphenyl)-2-[3-(4-cyanophenyl)-2-oxo-1,4-diazaspiro[4.5]dec-3-en-1-yl]acetamide | 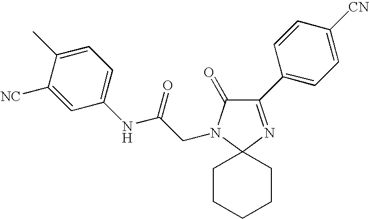 | Found 426 (MH⁺).C₂₅H₂₃N₅O₂Requires 425.5;3.18 | B |

### Example 59

### 2-[3-(4-cyanophenyl)-2-oxo-1,4-diazaspiro[4.5]dec-3-en-1-yl]-N-(3,5-difluorophenyl)acetamide

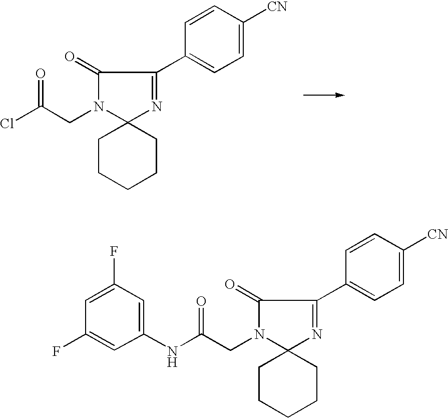

The title compound (148 mg) was prepared from [3-(4-cyanophenyl)-2-oxo-1,4-diazaspiro[4.5]dec-3-en-1-yl]acetyl chloride D40 (201 mg 0.63 mmol)) and 3,5-difluoroaniline (98 mg) using a similar method to Example 1. Purification was by silica gel chromatography used 0-50% ethyl acetate in hexane.

¹H NMR (CDCl₃) δ: 1.28-1.39 (3H, m), 1.84-2.11 (7H, m), 4.24 (2H, s), 6.56 (1H, m), 7.12 (2H, m), 7.80 (2H, d), 8.60 (2H, d), 9.04 (1H, br s).

Mass Spectrum (Electrospray LC/MS) Found 423 (MH⁺). C₂₃H₂₀F₂N₄O₂ requires 422. Ret. Time 3.44 min.

Examples 60-69 below can be made following similar procedures to the preparation of Example 15 or of Example 1.

| Example no. | Structure | Name |
|---|---|---|
| 60 | 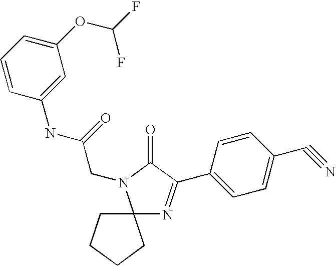 | 2-[3-(4-cyanophenyl)-2-oxo-1,4-diazaspiro[4.4]non-3-en-1-yl]-N-{3-[(difluoromethyl)oxy]phenyl}acetamide |
| 61 | 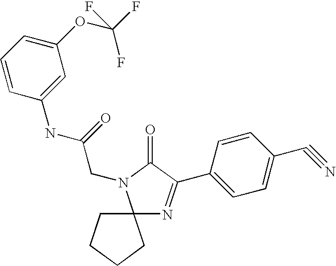 | 2-[3-(4-cyanophenyl)-2-oxo-1,4-diazaspiro[4.4]non-3-en-1-yl]-N-{3-[(trifluoromethyl)oxy]phenyl}acetamide |
| 62 | 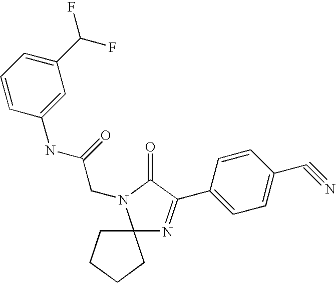 | 2-[3-(4-cyanophenyl)-2-oxo-1,4-diazaspiro[4.4]non-3-en-1-yl]-N-[3-(difluoromethyl)phenyl]acetamide |
| 63 | 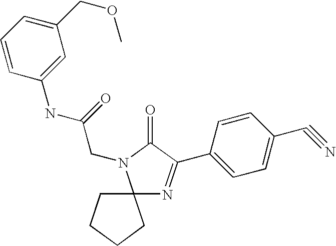 | 2-[3-(4-cyanophenyl)-2-oxo-1,4-diazaspiro[4.4]non-3-en-1-yl]-N-{3-[(methyloxy)methyl]phenyl}acetamide |
| 64 | 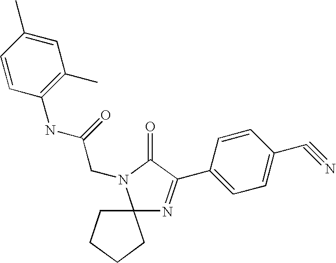 | 2-[3-(4-cyanophenyl)-2-oxo-1,4-diazaspiro[4.4]non-3-en-1-yl]-N-(2,4-dimethylphenyl)acetamide |
| 65 | 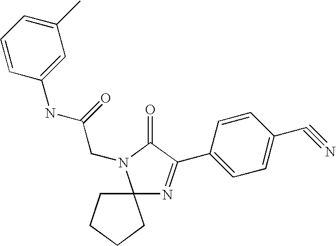 | 2-[3-(4-cyanophenyl)-2-oxo-1,4-diazaspiro[4.4]non-3-en-1-yl]-N-(3-methylphenyl)acetamide |
| 66 | 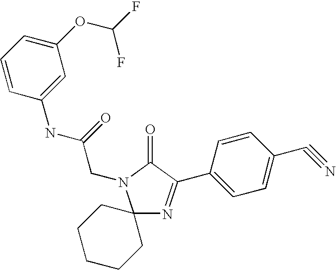 | 2-[3-(4-cyanophenyl)-2-oxo-1,4-diazaspiro[4.5]dec-3-en-1-yl]-N-{3-[(difluoromethyl)oxy]phenyl}acetamide |
| 67 | 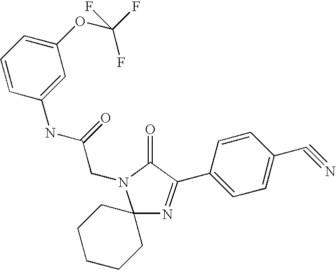 | 2-[3-(4-cyanophenyl)-2-oxo-1,4-diazaspiro[4.5]dec-3-en-1-yl]-N-{3-[(trifluoromethyl)oxy]phenyl}acetamide |
| 68 | 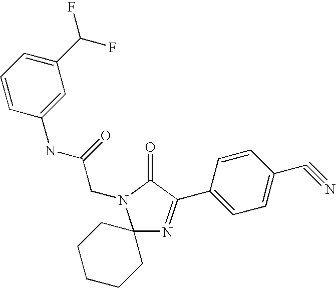 | 2-[3-(4-cyanophenyl)-2-oxo-1,4-diazaspiro[4.5]dec-3-en-1-yl]-N-[3-(difluoromethyl)phenyl]acetamide |
| 69 | 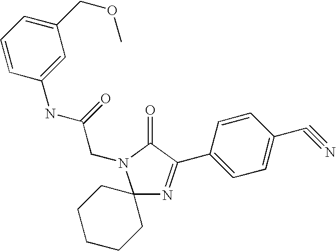 | 2-[3-(4-cyanophenyl)-2-oxo-1,4-diazaspiro[4.5]dec-3-en-1-yl]-N-{3-[(methyloxy)methyl]phenyl}acetamide |

1-22. (canceled)

23. A compound of formula (I) or a salt or solvate thereof: 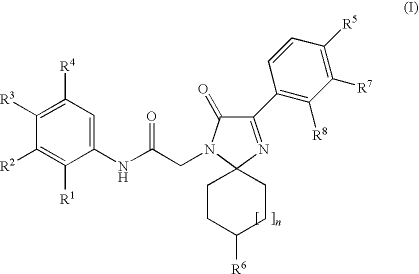
wherein:
R¹ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl or cyano;
R² is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl or cyano;
R³ is H, methyl, ethyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl or cyano;
or R² and R³ together form a group which is —O—CH₂—O— or —O—CH₂—CH₂—O—;
R⁴ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl or cyano;
R⁵ is H, C₁-C₄alkyl, C₁-C₄alkoxy, haloC₁-C₄alkyl, haloC₁-C₄alkoxy, halo, cyano, or C₁-C₄alkoxyC₁-C₄alkoxy;
R⁷ is H, C₁-C₄alkyl, C₁-C₄alkoxy, haloC₁-C₄alkyl, haloC₁-C₄alkoxy, halo, cyano, or C₁-C₄alkoxyC₁-C₄alkoxy;
with the proviso that when R⁵ is H, methyl, methoxy, chloro and fluoro, then R⁷ is not H;
R⁶ is H or methyl;
R⁸ is H or fluoro; and
n is 0, 1 or 2.

24. A compound as claimed in claim 23 wherein R¹ is H, C₁-C₂alkyl, C₁-C₂alkoxy, halo, haloC₁-C₂alkyl, haloC₁-C₂alkoxy, or cyano.

25. A compound as claimed in claim 23 wherein R² is H, C₁-C₂alkyl, C₁-C₂alkoxy, halo, haloC₁-C₂alkyl, haloC₁-C₂alkoxy, or cyano.

26. A compound as claimed in claim 23 wherein R³ is H, C₁-C₂alkyl, C₁-C₂alkoxy, halo, haloC₁-C₂alkyl, haloC₁-C₂alkoxy, or cyano.

27. A compound as claimed in claim 1 wherein R⁴ is H, C₁-C₂alkyl, C₁-C₂alkoxy, halo, haloC₁-C₂alkyl, haloC₁-C₂alkoxy, or cyano.

28. A compound as claimed in claim 1 wherein R⁵ is H, C₁-C₂alkoxy, haloC₁-C₂alkyl, haloC₁-C₂alkoxy, halo, cyano, or C₁-C₂alkoxyC₁-C₂alkoxy.

29. A compound as claimed in claim 6, wherein R⁵ is H, trifluoromethyl, trifluoromethoxy, bromo, cyano, or methoxyethoxy.

30. A compound as claimed in claim 1 wherein R⁵ is H, methyl, methoxy, chloro or fluoro, and R⁷ is C₁-C₂alkyl, C₁-C₂alkoxy, haloC₁-C₂alkyl, haloC₁-C₂alkoxy, halo, cyano, or C₁-C₂alkoxyC₁-C₂alkoxy.

31. A compound as claimed in claim 1 wherein R⁷ is C₁-C₂alkyl, C₁-C₂alkoxy, haloC₁-C₂alkyl, haloC₁-C₂alkoxy, halo, cyano, or C₁-C₂alkoxyC₁-C₂alkoxy.

32. A compound as claimed in claim 1 wherein R⁵ is H, haloC₁-C₄alkyl, haloC₁-C₄alkoxy, bromo, cyano, or C₁-C₄alkoxyC₁-C₄alkoxy; and R⁷ is H, C₁-C₄alkyl, C₁-C₄alkoxy, haloC₁-C₄alkyl, haloC₁-C₄alkoxy, halo, cyano, or C₁-C₄alkoxyC₁-C₄alkoxy; and R⁵ and R⁷ are not both H.

33. A compound as claimed in claim 1 wherein R⁶ is H.

34. A compound as claimed in claim 1 wherein n is 0 or 1.

35. A compound as claimed in claim 1 which is:
N-[2-(methyloxy)phenyl]-2-{2-oxo-3-[4-(trifluoromethyl)phenyl]-1,4-diazaspiro[4.5]dec-3-en-1-yl}acetamide;
2-[3-(4-bromophenyl)-2-oxo-1,4-diazaspiro[4.5]dec-3-en-1-yl]-N-(3,4-difluorophenyl)acetamide;
2-[3-(4-cyanophenyl)-2-oxo-1,4-diazaspiro[4.5]dec-3-en-1-yl]-N-(3,4-difluorophenyl)acetamide;
N-(3-methylphenyl)-2-{2-oxo-3-[4-(trifluoromethyl)phenyl]-1,4-diazaspiro[4.5]dec-3-en-1-yl}acetamide;
N-(3,4-difluorophenyl)-2-{2-oxo-3-[4-(trifluoromethyl)phenyl]-1,4-diazaspiro[4.5]dec-3-en-1-yl}acetamide;
N-(3,4-difluorophenyl)-2-(2-oxo-3-{4-[(trifluoromethyl)oxy]phenyl}-1,4-diazaspiro[4.5]dec-3-en-1-yl)acetamide;
N-(2,4-dimethylphenyl)-2-(2-oxo-3-{4-[(trifluoromethyl)oxy]phenyl}-1,4-diazaspiro[4.5]dec-3-en-1-yl)acetamide;
N-[2-(methyloxy)phenyl]-2-(2-oxo-3-{4-[(trifluoromethyl)oxy]phenyl}-1,4-diazaspiro[4.5]dec-3-en-1-yl)acetamide;
2-[3-(3-chlorophenyl)-2-oxo-1,4-diazaspiro[4.5]dec-3-en-1-yl]-N-(3,4-difluorophenyl)acetamide;
2-[3-(3-chlorophenyl)-2-oxo-1,4-diazaspiro[4.5]dec-3-en-1-yl]-N-(2-methylphenyl)acetamide;
2-[3-(3-chlorophenyl)-2-oxo-1,4-diazaspiro[4.5]dec-3-en-1-yl]-N-(2,4-dimethylphenyl)acetamide;
2-[3-(3-chlorophenyl)-2-oxo-1,4-diazaspiro[4.5]dec-3-en-1-yl]-N-[2-(methyloxy)phenyl]acetamide;
2-[3-(4-bromophenyl)-2-oxo-1,4-diazaspiro[4.5]dec-3-en-1-yl]-N-(2,4-dimethylphenyl)acetamide;
N-(3,4-difluorophenyl)-2-[3-(4-{[2-(methyloxy)ethyl]oxy}phenyl)-2-oxo-1,4-diazaspiro[4.5]dec-3-en-1-yl]acetamide;
2-[3-(4-cyanophenyl)-2-oxo-1,4-diazaspiro[4.4]non-3-en-1-yl]-N-(3,5-difluorophenyl)acetamide;
N-(2-chlorophenyl)-2-{2-oxo-3-[4-(trifluoromethyl)phenyl]-1,4-diazaspiro[4.4]non-3-en-1-yl}acetamide;
N-(3,4-difluorophenyl)-2-{2-oxo-3-[4-(trifluoromethyl)phenyl]-1,4-diazaspiro[4.4]non-3-en-1-yl}acetamide;
N-(3,5-difluorophenyl)-2-{2-oxo-3-[4-(trifluoromethyl)phenyl]-1,4-diazaspiro[4.4]non-3-en-1-yl}acetamide;
N-[3,5-bis(trifluoromethyl)phenyl]-2-{2-oxo-3-[4-(trifluoromethyl)phenyl]-1,4-diazaspiro[4.5]dec-3-en-1-yl}acetamide;
N-[3-cyano-5-(trifluoromethyl)phenyl]-2-{2-oxo-3-[4-(trifluoromethyl)phenyl]-1,4-diazaspiro[4.5]dec-3-en-1-yl}acetamide;
N-[3-bromo-5-(trifluoromethyl)phenyl]-2-{2-oxo-3-[4-(trifluoromethyl)phenyl]-1,4-diazaspiro[4.5]dec-3-en-1-yl}acetamide;
N-(4-chloro-3-methylphenyl)-2-{2-oxo-3-[4-(trifluoromethyl)phenyl]-1,4-diazaspiro[4.5]dec-3-en-1-yl}acetamide;
N-(3-fluoro-5-methylphenyl)-2-{2-oxo-3-[4-(trifluoromethyl)phenyl]-1,4-diazaspiro[4.5]dec-3-en-1-yl}acetamide;
N-(2-fluoro-5-methylphenyl)-2-{2-oxo-3-[4-(trifluoromethyl)phenyl]-1,4-diazaspiro[4.5]dec-3-en-1-yl}acetamide;
N-(4-fluoro-3-methylphenyl)-2-{2-oxo-3-[4-(trifluoromethyl)phenyl]-1,4-diazaspiro[4.5]dec-3-en-1-yl}acetamide;
2-[3-(4-cyanophenyl)-2-oxo-1,4-diazaspiro[4.5]dec-3-en-1-yl]-N-(3,5-difluorophenyl)acetamide;
N-(3,5-difluorophenyl)-2-(2-oxo-3-{4-[(trifluoromethyl)oxy]phenyl}-1,4-diazaspiro[4.5]dec-3-en-1-yl)acetamide;
2-(2-oxo-3-{4-[(trifluoromethyl)oxy]phenyl}-1,4-diazaspiro[4.5]dec-3-en-1-yl)-N-(3,4,5-trifluorophenyl)acetamide;
2-(2-oxo-3-{4-[(trifluoromethyl)oxy]phenyl}-1,4-diazaspiro[4.5]dec-3-en-1-yl)-N-(2,3,5-trifluorophenyl)acetamide;
2-[3-(4-cyanophenyl)-2-oxo-1,4-diazaspiro[4.5]dec-3-en-1-yl]-N-(3,4,5-trifluorophenyl)acetamide;
N-(4-bromo-3,5-difluorophenyl)-2-[3-(4-cyanophenyl)-2-oxo-1,4-diazaspiro[4.5]dec-3-en-1-yl]acetamide;
N-(2-chloro-3,5-difluorophenyl)-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.5]dec-3-en-1-yl]-N-[3,5-difluoro-4-(trifluoromethyl)phenyl]acetamide;
2-[3-(4-cyanophenyl)-2-oxo-1,4-diazaspiro[4.5]dec-3-en-1-yl]-N-[3,5-difluoro-4-(methyloxy)phenyl]acetamide;
N-(4-cyano-3,5-difluorophenyl)-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.4]non-3-en-1-yl]-N-(3,4-difluorophenyl)acetamide;
2-[3-(4-cyanophenyl)-2-oxo-1,4-diazaspiro[4.5]dec-3-en-1-yl]-N-(2,4-dimethylphenyl)acetamide;
2-{3-[3-bromo-4-(methyloxy)phenyl]-2-oxo-1,4-diazaspiro[4.5]dec-3-en-1-yl}-N-(3,5-difluorophenyl)acetamide;
2-{3-[3-cyano-4-(methyloxy)phenyl]-2-oxo-1,4-diazaspiro[4.5]dec-3-en-1-yl}-N-(3,5-difluorophenyl)acetamide;
2-[3-(4-cyanophenyl)-2-oxo-1,4-diazaspiro[4.4]non-3-en-1-yl]-N-[3-(trifluoromethyl)phenyl]acetamide;
2-[3-(4-cyanophenyl)-2-oxo-1,4-diazaspiro[4.5]dec-3-en-1-yl]-N-[3-(trifluoromethyl)phenyl]acetamide;
2-[3-(4-cyanophenyl)-2-oxo-1,4-diazaspiro[4.4]non-3-en-1-yl]-N-[4-fluoro-3-(trifluoromethyl)phenyl]acetamide;
N-[2-chloro-3-(trifluoromethyl)phenyl]-2-[3-(4-cyanophenyl)-2-oxo-1,4-diazaspiro[4.4]non-3-en-1-yl]acetamide;
N-[4-chloro-3-(trifluoromethyl)phenyl]-2-[3-(4-cyanophenyl)-2-oxo-1,4-diazaspiro[4.4]non-3-en-1-yl]acetamide;
2-[3-(4-cyanophenyl)-2-oxo-1,4-diazaspiro[4.5]dec-3-en-1-yl]-N-[4-fluoro-3-(trifluoromethyl)phenyl]acetamide;
N-[2-chloro-3-(trifluoromethyl)phenyl]-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.4]non-3-en-1-yl]-N-[3-fluoro-5-(trifluoromethyl)phenyl]acetamide;
2-[3-(4-cyanophenyl)-2-oxo-1,4-diazaspiro[4.5]dec-3-en-1-yl]-N-[3-fluoro-5-(trifluoromethyl)phenyl]acetamide;
2-[3-(4-cyanophenyl)-2-oxo-1,4-diazaspiro[4.4]non-3-en-1-yl]-N-[2-methyl-5-(trifluoromethyl)phenyl]acetamide;
2-[3-(4-cyanophenyl)-2-oxo-1,4-diazaspiro[4.5]dec-3-en-1-yl]-N-[2-methyl-5-(trifluoromethyl)phenyl]acetamide;
N-[4-chloro-3-(trifluoromethyl)phenyl]-2-[3-(4-cyanophenyl)-2-oxo-1,4-diazaspiro[4.5]dec-3-en-1-yl]acetamide;
N-[2-chloro-5-(trifluoromethyl)phenyl]-2-[3-(4-cyanophenyl)-2-oxo-1,4-diazaspiro[4.4]non-3-en-1-yl]acetamide;
N-[2-chloro-5-(trifluoromethyl)phenyl]-2-[3-(4-cyanophenyl)-2-oxo-1,4-diazaspiro[4.5]dec-3-en-1-yl]acetamide;
N-(3-cyanophenyl)-2-[3-(4-cyanophenyl)-2-oxo-1,4-diazaspiro[4.4]non-3-en-1-yl]acetamide;
N-(3-cyanophenyl)-2-[3-(4-cyanophenyl)-2-oxo-1,4-diazaspiro[4.5]DEC-3-en-1-yl]acetamide;
N-(3-cyano-4-fluorophenyl)-2-[3-(4-cyanophenyl)-2-oxo-1,4-diazaspiro[4.5]dec-3-en-1-yl]acetamide;
N-(3-cyano-5-fluorophenyl)-2-[3-(4-cyanophenyl)-2-oxo-1,4-diazaspiro[4.5]dec-3-en-1-yl]acetamide;
N-(3-cyano-4-methylphenyl)-2-[3-(4-cyanophenyl)-2-oxo-1,4-diazaspiro[4.5]dec-3-en-1-yl]acetamide;
2-[3-(4-cyanophenyl)-2-oxo-1,4-diazaspiro[4.4]non-3-en-1-yl]-N-{3-[(difluoromethyl)oxy]phenyl}acetamide;
2-[3-(4-cyanophenyl)-2-oxo-1,4-diazaspiro[4.4]non-3-en-1-yl]-N-{3-[(trifluoromethyl)oxy]phenyl}acetamide;
2-[3-(4-cyanophenyl)-2-oxo-1,4-diazaspiro[4.4]non-3-en-1-yl]-N-[3-(difluoromethyl)phenyl]acetamide;
2-[3-(4-cyanophenyl)-2-oxo-1,4-diazaspiro[4.4]non-3-en-1-yl]-N-{3-[(methyloxy)methyl]phenyl}acetamide;
2-[3-(4-cyanophenyl)-2-oxo-1,4-diazaspiro[4.4]non-3-en-1-yl]-N-(2,4-dimethylphenyl)acetamide;
2-[3-(4-cyanophenyl)-2-oxo-1,4-diazaspiro[4.4]non-3-en-1-yl]-N-(3-methylphenyl)acetamide;
2-[3-(4-cyanophenyl)-2-oxo-1,4-diazaspiro[4.5]dec-3-en-1-yl]-N-{3-[(difluoromethyl)oxy]phenyl}acetamide;
2-[3-(4-cyanophenyl)-2-oxo-1,4-diazaspiro[4.5]dec-3-en-1-yl]-N-{3-[(trifluoromethyl)oxy]phenyl}acetamide;
2-[3-(4-cyanophenyl)-2-oxo-1,4-diazaspiro[4.5]dec-3-en-1-yl]-N-[3-(difluoromethyl)phenyl]acetamide;
2-[3-(4-cyanophenyl)-2-oxo-1,4-diazaspiro[4.5]dec-3-en-1-yl]-N-{3-[(methyloxy)methyl]phenyl}acetamide;
or a salt or solvate thereof.

36. A method of treating schizophrenia, dementia or attention deficit disorder in a patient suffering from such a disorder comprising administering an effective amount of a compound of formula 1 or a salt thereof according to claim 1.

37. A pharmaceutical composition comprising a compound as claimed 1 or a salt thereof and at least one pharmaceutically acceptable carrier, diluent or excipient.

38. A process for the manufacture of a compound as claimed in claim 23, the process comprising:
(a) reacting a compound of formula (II): 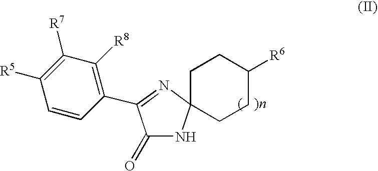
wherein R⁵, R⁷, R⁶, R⁸ and n are as defined for formula (I), with a compound of formula (III): 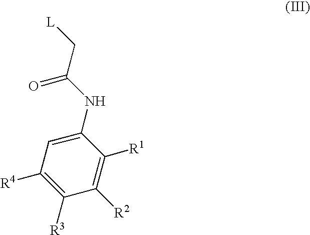
wherein R¹, R², R³, R⁴ and are as defined for formula (I), and L is a leaving group;
or
(b) reacting a compound of formula (XV): 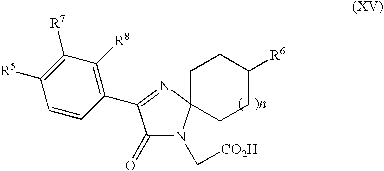
wherein R⁵, R⁷, R⁶, R⁸ and n are as defined for formula (I), with a compound of formula (IV): 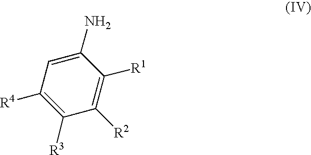
wherein R¹, R², R³ and R⁴ are as defined for formula (I);
or
(c) reacting a compound of formula (XVI): 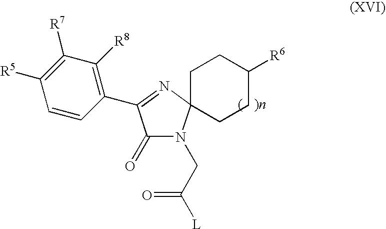
wherein R⁵, R⁶, R⁷ and n are as defined in formula (I) and L represents a leaving group; with a compound of formula (IV) as defined in process (b);
and thereafter optionally:
removing any protecting groups and/or
converting a compound as claimed in claim 1 into another compound as claimed in claim 23 and/or
forming a salt or solvate.
